SEQUENCE LISTING (29 sequences)

SEQ ID NO: 1 - protein, 4 aa, Artificial Sequence
  Description of Artificial Sequence Synthetic peptide
LQGV

SEQ ID NO: 2 - protein, 4 aa, Artificial Sequence
  Description of Artificial Sequence Synthetic peptide
AQGV

SEQ ID NO: 3 - protein, 4 aa, Artificial Sequence
  Description of Artificial Sequence Synthetic peptide
LQGA

SEQ ID NO: 4 - protein, 6 aa, Artificial Sequence
  Description of Artificial Sequence Synthetic peptide
VLPALP

SEQ ID NO: 5 - protein, 6 aa, Artificial Sequence
  Description of Artificial Sequence Synthetic peptide
ALPALP

SEQ ID NO: 6 - protein, 6 aa, Artificial Sequence
  Description of Artificial Sequence Synthetic peptide
VAPALP

SEQ ID NO: 7 - protein, 7 aa, Artificial Sequence
  Description of Artificial Sequence Synthetic peptide
ALPALPQ

SEQ ID NO: 8 - protein, 7 aa, Artificial Sequence
  Description of Artificial Sequence Synthetic peptide
VLPAAPQ

SEQ ID NO: 9 - protein, 7 aa, Artificial Sequence
  Description of Artificial Sequence Synthetic peptide
VLPALAQ

SEQ ID NO: 10 - protein, 4 aa, Artificial Sequence
  Description of Artificial Sequence Synthetic peptide
LAGV

SEQ ID NO: 11 - protein, 6 aa, Artificial Sequence
  Description of Artificial Sequence Synthetic peptide
VLAALP

SEQ ID NO: 12 - protein, 6 aa, Artificial Sequence
  Description of Artificial Sequence Synthetic peptide
VLPALA

SEQ ID NO: 13 - protein, 7 aa, Artificial Sequence
  Description of Artificial Sequence Synthetic peptide
VLPALPQ

SEQ ID NO: 14 - protein, 7 aa, Artificial Sequence
  Description of Artificial Sequence Synthetic peptide
VLAALPQ

SEQ ID NO: 15 - protein, 7 aa, Artificial Sequence
  Description of Artificial Sequence Synthetic peptide
VLPALPA

SEQ ID NO: 16 - protein, 7 aa, Artificial Sequence
  Description of Artificial Sequence Synthetic peptide
GVLPALP

SEQ ID NO: 17 - protein, 13 aa, Artificial Sequence
  Description of Artificial Sequence Synthetic peptide
LQGVLPALPQVVC

SEQ ID NO: 18 - protein, 13 aa, Artificial Sequence
  Description of Artificial Sequence Synthetic peptide
LPGCPRGVNPVVS

SEQ ID NO: 19 - protein, 4 aa, Artificial Sequence
  Description of Artificial Sequence Synthetic peptide
LPGC

SEQ ID NO: 20 - protein, 4 aa, Artificial Sequence
  Description of Artificial Sequence Synthetic peptide
MTRV

SEQ ID NO: 21 - protein, 10 aa, Artificial Sequence
  Description of Artificial Sequence Synthetic peptide
VLPALPQVVC

SEQ ID NO: 22 - protein, 10 aa, Artificial Sequence
  Description of Artificial Sequence Synthetic peptide
LQGVLPALPQ

SEQ ID NO: 23 - protein, 8 aa, Artificial Sequence
  Description of Artificial Sequence Synthetic peptide
GVLPALPQ

SEQ ID NO: 24 - protein, 38 aa, Artificial Sequence
  Description of Artificial Sequence Synthetic peptide
VVCNYRDVRFESIRLPGCPRGVNPVVSYAVALSCQCAL

SEQ ID NO: 25 - protein, 35 aa, Artificial Sequence
  Description of Artificial Sequence Synthetic peptide
RPRCRPINATLAVEKEGCPVCITVNTTICAGYCPT

SEQ ID NO: 26 - protein, 18 aa, Artificial Sequence
  Description of Artificial Sequence Synthetic peptide
SKAPPPSLPSPSRLPGPS

SEQ ID NO: 27 - protein, 16 aa, Artificial Sequence
  Description of Artificial Sequence Synthetic peptide
SIRLPGCPRGVNPVVS

SEQ ID NO: 28 - DNA, 25 bp, Artificial Sequence
  Description of Artificial Sequence Synthetic probe
agctcagagggggactttccgagag

SEQ ID NO: 29 - protein, 4 aa, Artificial Sequence
  Description of Artificial Sequence Synthetic peptide
QVVC

(19) United States
(12) Patent Application Publication
(10) Pub. No.: US 2008/0318871 A1
(43) Pub. Date: Dec. 25, 2008

(54) Treatment of neurological disorders

(76) Inventors: Nisar A. Khan, Rotterdam (NL); Gert Wensvoort, Koekange (NL); Robert Benner, Barendrecht (NL); Leonie A. Boven, Rotterdam (NL)

Correspondence Address: TRASK BRITT, P.O. BOX 2550, SALT LAKE CITY, UT, 84110, US

(21) Appl. No.: 11/982,292

(22) Filed: Oct. 31, 2007

Related U.S. Application Data
(63) Continuation-in-part of application No. 10/409,654, filed on Apr. 8, 2003, now abandoned.
Application No. 10/409,654 is a continuation-in-part of application No. 10/028,075, filed on Dec. 21, 2001.

(51) Int. Cl. A61K 38/08; A61K 38/07; A61P 25/00; A61K 38/06

(52) U.S. Cl. 514/16; 514/18; 514/17

(57) ABSTRACT

The description relates to the treatment of the inflammatory component of neurological disorders or so called neuroimmune disorders such as schizophrenia, manic depression and other bipolar disorders, multiple sclerosis, post-partum psychosis and autism, herein also called inflammatory neurological disorders. Provided are methods for modulating a neurological disorder in a subject comprising providing the subject with a gene-regulatory peptide or functional analogue thereof. Also provided is use of an NF-kappaB down-regulating peptide or functional analogue thereof for the production of a pharmaceutical composition for the treatment of a neurological disorder.

### CROSS-REFERENCE TO RELATED APPLICATIONS

This application is a continuation-in-part of co-pending U.S. patent application Ser. No. 10/409,654, filed Apr. 8, 2003, now U.S. Pat. No. ______, which is a continuation-in-part of U.S. patent application Ser. No. 10/028,075, filed Dec. 21, 2001 now U.S. Pat. No. ______, and of U.S. patent application Ser. No. 11/346,761, filed Feb. 3, 2006, now U.S. Pat. No. ______, which is a continuation of U.S. patent application Ser. No. 11/286,571, filed Nov. 23, 2005, now U.S. Pat. No. ______, which is a continuation-in-part of U.S. patent application Ser. No. 10/409,654, filed Apr. 8, 2003, now U.S. Pat. No. ______, which is a continuation-in-part of U.S. patent application Ser. No. 10/028,075, filed Dec. 21, 2001 now U.S. Pat. No. ______, the contents of the entirety of each of which are hereby incorporated herein by this reference.

### TECHNICAL FIELD

The current invention relates generally to biotechnology, and, more particularly, to the body's innate way of modulating important physiological processes and builds on insights reported in WO 99/59717, WO 01/00259 and PCT/NL/00639, the contents of the entirety of each of which are hereby incorporated herein by this reference. In particular, the invention relates to the treatment of the inflammatory component of neurological disorders or so called neuroimmune disorders such as schizophrenia, manic depression, and other bipolar disorders, multiple sclerosis, post-partum psychosis, and autism, herein also called inflammatory neurological disorders.

### BACKGROUND

In these earlier patent applications, small gene-regulatory peptides were described that are present naturally in pregnant women and are derived from proteolytic breakdown of placental gonadotropins such as human chorionic gonadotropin (hCG) produced during pregnancy. These peptides (in their active state often only at about four to six amino acids long) were shown to have unsurpassed immunological activity that they exert by regulating expression of genes encoding inflammatory mediators such as cytokines. Surprisingly, it was found that breakdown of hCG provides a cascade of peptides that help maintain a pregnant woman's immunological homeostasis. These peptides are nature's own substances that balance the immune system to assure that the mother stays immunologically sound while her fetus does not get prematurely rejected during pregnancy but instead is safely carried through its time of birth.

Where it was generally thought that the smallest breakdown products of proteins had no specific biological function on their own (except to potentially serve as an antigen for the immune system), it now emerges that the body, in fact, routinely utilizes the normal process of proteolytic breakdown of the proteins it produces to generate important gene-regulatory compounds, short peptides that control the expression of the body's own genes. Apparently, the body uses a gene-control system affected by small breakdown products of the exact proteins that are encoded by its own genes.

During pregnancy, the maternal system introduces a status of temporary immuno-modulation which results in suppression of maternal rejection responses directed against the fetus. Paradoxically, during pregnancy, often the mother's resistance to infection is increased and she is found to be better protected against the clinical symptoms of various auto-immune diseases such as rheumatism and multiple sclerosis. The protection of the fetus can thus not be interpreted only as a result of immune suppression. Each of the above three applications have provided insights by which the immunological balance between protection of the mother and protection of the fetus can be understood.

Inventors hereof have earlier shown that certain short breakdown products of hCG (i.e., short peptides which can easily be synthesized, if need be modified, and used as pharmaceutical composition) exert a major regulatory activity on pro- or anti-inflammatory cytokine cascades that are governed by a family of crucial transcription factors, the NFkappaB family which stands central in regulating the expression of genes that shape the body's immune response.

Most of the hCG produced during pregnancy is produced by cells of the placenta, the exact organ where cells and tissues of mother and child most intensely meet and where immuno-modulation is most needed to fight off rejection. Being produced locally, the gene-regulatory peptides which are broken down from hCG in the placenta immediately balance the pro- or anti-inflammatory cytokine cascades found in the no-mans land between mother and child. Being produced by the typical placental cell, the trophoblast, the peptides traverse extracellular space; enter cells of the immune system and exert their immuno-modulatory activity by modulating NFkappaB-mediated expression of cytokine genes, thereby keeping the immunological responses in the placenta at bay.

### DISCLOSURE OF THE INVENTION

The beneficial effects seen on the occurrence and severity of auto-immune disease in the pregnant woman result from an overspill of the hCG-derived peptides into the body as a whole; however, these effects must not be overestimated, as it is easily understood that the further away from the placenta, the less immuno-modulatory activity aimed at preventing rejection of the fetus will be seen, if only because of a dilution of the placenta-produced peptides throughout the body as a whole. However, the immuno-modulatory and gene-regulatory activity of the peptides should by no means only be thought to occur during pregnancy and in the placenta; man and women alike produce hCG, for example in their pituitaries, and nature certainly utilizes the gene-regulatory activities of peptides in a larger whole.

Consequently, a novel therapeutic inroad is provided, using the pharmaceutical potential of gene-regulatory peptides and derivatives thereof. Indeed, evidence of specific up- or down-regulation of NFkappaB driven pro- or anti-inflammatory cytokine cascades that are each, and in concert, directing the body's immune response was found in silico in gene-arrays by expression profiling studies, in vitro after treatment of immune cells and in vivo in experimental animals treated with gene-regulatory peptides. Also, considering that NFkappaB is a primary effector of disease (A. S. Baldwin, J. Clin. Invest., 2001, 107:3-6), using the hCG derived gene-regulatory peptides offer significant potential for the treatment of a variety of human and animal diseases, thereby tapping the pharmaceutical potential of the exact substances that help balance the mother's immune system such that her pregnancy is safely maintained.

### DETAILED DESCRIPTION OF THE INVENTION

The disclosure hereof, in particular, relates to the treatment of neurological disorders or so called neuroimmune disorders such as schizophrenia, manic depression and other bipolar disorders, multiple sclerosis, post-partum psychosis, autism, chronic fatigue syndrome (CFS), fibromyalgia, Alzheimers, mood disorders and certain forms of stress, and in particular to those neurological disorders in which local brain inflammatory processes are involved. Provided is peptide compounds that have beneficial activity on anti-inflammatory M2-type macrophages and actively contribute to the resolution of brain inflammation and hence to tissue integrity and function. Although there are major differences in etiology and mechanisms of pathogenesis of each of these syndromes and or diseases, there are, in fact, common inflammatory and immunomodulatory pathways that are shared within the pathogenesis of neurological disorders.

Evidence of immune abnormalities in patients suffering from psychological disease clearly shows the implication of the immune system in pathogenesis. Neuroimmune disorders have become recognized as common pathogenetic factors in the development of psycho- or neuropathologies. The neurochemical and immunologic findings indicate multiple pathways of the pathogenesis; herein, we discuss the role of inflammatory disease in neurological disorders. For example, chronic fatigue syndrome is a condition that affects women in disproportionate numbers, and that is often exacerbated in the premenstrual period and following physical exertion. The signs and symptoms, which include fatigue, myalgia, and low-grade fever, are similar to those experienced by patients infused with cytokines such as interleukin-1.

In general, during the development of a neuroimmune disorder, the TNF-alpha family and other pro-inflammatory cytokines are highly elevated in cerebrospinal fluid (CSF), demonstrative of foci of inflammation in the brain leading to an array of destructive and degenerative responses directed at diverse areas in the CNS. Major mood disorders are leading causes of disability from early adolescence onward and leading sources of disease burden, surpassing cardiovascular diseases, dementia, lung cancer and diabetes. As said, there is a major role for inflammatory cytokines and immune cells in the pathophysiology of mood disorders, it was recently also found that T cells and monocytes function at a higher, pro-inflammatory level in patients with bipolar disorder. Successful therapy of these destructive and degenerative disorders that affect the adult human central nervous system (CNS) will require the ability both to reduce the rate and extent of tissue injury, and to restore or replace destroyed tissue. Neuroimaging studies have shown that functional organization occurs spontaneously in the adult human brain in response to tissue insults. The extent of this compensatory mechanism may be limited, necessitating development of active methods of intervention. Replacement of a single neurotransmitter, neurohormone or trophic factor may suffice if the injury is limited or affected as a suppressed or altered pathway within the CNS through proinflammatory regulators. The hippocampus is a source for mitotically active neuronal progenitor cells which can hypothetically replace neurons and myelinating cells. It is the control of these cells and the health and activity of other cells which offers new insight and hope of treating heretofore chronic CNS disease.

It is areas such as cells in the adult human dentate gyrus which may be part of the key to controlling immunomodulation and growth support of the brain and its diverse functions which span from memory and cognition to its endocrine and immunologic activities. As with all complex traits, a neurological disorder results from interplay between as yet unidentified environmental factors and susceptibility genes. Together, these factors trigger a cascade of events, involving engagement of the immune system, acute inflammatory injury of the central nervous system, notably axons and glia, recovery of function and structural repair, post-inflammatory gliosis, and neurodegeneration. The sequential involvement of these processes underlies the clinical course characterized by episodes with recovery interchanged with episodes leaving persistent deficits, episodes which we generally call psychological disorders.

For a more detailed example, although there are several forms of autism (which often present themselves already at birth), which may have clear genetic etiologies, the most common forms however occur long after normal births and are associated with proinflammatory cytokine dysregulation. According to recent epidemiological surveys, autistic spectrum disorders have become recognized as common childhood psychopathologies. These lifelong conditions demonstrate a strong genetic determinant consistent with a polygenic mode of inheritance for which several autism susceptibility regions have been identified. Parallel evidence of immune abnormalities in autistic patients argues for an implication of the immune system in pathogenesis. This introduction summarizes advances in the molecular genetics of autism, as well as recently emerging concerns addressing the disease incidence and triggering factors. The neurochemical and immunologic findings are analyzed in the context of a neuroimmune hypothesis for specific neurological disorders. For example, pregnancy and the post partum period are important modulators of the immune system and the immune suppression in pregnancy is followed by an immune activation in the puerperium.

In another example, autism appears to be influenced by specific food allergies or even the early use of vaccines which may cause changes in the regulation of innate or acquired immunity and set up neuroendocrine dysfunction. Also, neurological disorders are often associated with autoimmune disorders in the patients' relatives. A. M. Comi et al. (J. Child Neurol. 1999 June; 14(6):388-94) evaluated the frequency of autoimmune disorders, as well as various prenatal and postnatal events in autism, and surveyed the families of 61 autistic patients and 46 healthy controls using questionnaires. The mean number of autoimmune disorders was greater in families with autism; 46% had two or more members with autoimmune disorders. As the number of family members with autoimmune disorders increased from one to three, the risk of autism was greater, with an odds ratio that increased from 1.9 to 5.5, respectively. In mothers and first-degree relatives of autistic children, there were more autoimmune disorders (16% and 21%) as compared to controls (2% and 4%), with odds ratios of 8.8 and 6.0, respectively. The most common autoimmune disorders in both groups were type 1 diabetes, adult rheumatoid arthritis, hypothyroidism, and systemic lupus erythematosus. Forty-six percent of the autism group reported having relatives with rheumatoid diseases, as compared to 26% of the controls. Prenatal maternal urinary tract, upper respiratory, and vaginal infections; asphyxia; prematurity, and seizures were more common in the autistic group, although the differences were not significant. Thirty-nine percent of the controls, but only 11% of the autistic, group, reported allergies.

The increased number of autoimmune disorders shows that in autism, immune dysfunction interacts with various environmental factors to play a role in autism pathogenesis. According to S. B. Edelson and D. S. Cantor (Toxicol. Ind. Health 1998 July-August; 14(4):553-63) the advances in medical technology during the last four decades have provided evidence for an underlying neurological basis for autism. The etiology for the variations of neurofunctional anomalies found in the neurological disorder spectrum behaviors appears inconclusive as of this date but growing evidence supports the proposal that chronic exposure to toxic agents, i.e., xenobiotic agents, resulting in a inflammatory reaction directed towards a developing central nervous system may be the best model for defining the physiological and behavioral data found in these populations. Also, an examination of 18 autistic children in blood analyses that were available showed that 16 of these children showed evidence of levels of toxic chemicals exceeding adult maximum tolerance. In the two cases where toxic chemical levels were not found, there was abnormal D-glucaric acid findings suggesting abnormal xenobiotic influences on liver detoxification processes.

A proposed mechanism for the interaction of xenobiotic toxins with immune system dysfunction and continuous and/or progressive endogenous toxicity is presented as it relates to the development of behaviors found in the autistic spectrum. H. Jyonouchi et al. (J. Neuroimmunol. 2001 Nov. 1; 120(1-2):170-9) determined innate and adaptive immune responses in children with developmental regression and autism spectrum disorders (ASD, N=71), developmentally normal siblings (N=23), and controls (N=17), and found a clear relationship between proinflammatory and regulatory cytokine production associated with innate and adaptive immune responses in children with autism spectrum disorders and developmental regression. With lipopolysaccharide (LPS), a stimulant for innate immunity, peripheral blood mononuclear cells (PBMCs) from 59/71 (83.1%) ASD patients produced >2 SD above the control mean (CM) values of TNF-alpha, IL-1beta, and/or IL-6 produced by control PBMCs. ASD PBMCs produced higher levels of proinflammatory/counter-regulatory cytokines without stimuli than controls. With stimulants of phytohemagglutinin (PHA), tetanus, IL-12p70, and IL-18, PBMCs from 47.9% to 60% of ASD patients produced >2 SD above the CM values of TNF-alpha depending on stimulants. These results indicate excessive innate immune responses as a result of NFkappaB induced cytokine expression in a number of ASD children that is most evident in TNF-alpha production. Furthermore, according to S. Messahel et al. (Neurosci. Lett. 1998 Jan. 23; 241(1):17-20), the pterins, neopterin and biopterin, occur naturally in body fluids including urine.

It is well established that increased neopterin levels are associated with activation of the cellular immune system and that reduced biopterins are essential for neurotransmitter synthesis. It has been also been suggested that some autistic children may be suffering from an autoimmune disorder. To investigate this further the above authors performed high performance liquid chromatography analyses of urinary pterins in a group of pre-school autistic children, their siblings and age-matched control children. Both urinary neopterin and biopterin were raised in the autistic children compared to controls and the siblings showed intermediate values.

As yet another example, the chronic fatigue syndrome (CFS) is a clinically defined condition characterized by severe disabling fatigue and a combination of symptoms that prominently features self-reported impairments in concentration and short-term memory, sleep disturbances, and musculoskeletal pain. Heretofore, the diagnosis of the chronic fatigue syndrome could only be made after other medical and psychiatric causes of chronic fatiguing illness were excluded. No pathognomonic signs or clear diagnostic tests for this condition have yet been validated. Thus far, no definitive treatment exists.

Recent longitudinal studies suggest that some persons affected by the chronic fatigue syndrome improve with time but that most remain functionally impaired for several years. CFS is characterized by debilitating fatigue that is not attributable to known clinical conditions, that has lasted for >six months, that has reduced the activity level of a previously healthy person by >50%, and that has been accompanied by flu-like symptoms (e.g., pharyngitis, adenopathy, low grade fever, myalgia, arthralgia, headache) and neuropsychological manifestations (e.g., difficulty concentrating, exercise intolerance, and sleep disturbances). CFS is frequently of sudden onset. There have been considerable advances in our understanding of the mediators of CFS, with several careful studies of immunologic function, activation, and cytokine dysregulation. An increasing number of independent groups have reported abnormalities of both T and B cell lymphocyte and NK cell function, with one group correlating levels of NK cell function to disease severity. It was suggested that the illness be named chronic immune activation syndrome given the abnormally elevated markers of T cell activation measured on T cells and cytotoxic T cells.

Over the last decade, investigators have demonstrated that individuals with CFS have significantly increased proportions of activated CD8+ T cells, decreased natural killer cell (NK) cytotoxic and lymphoproliferative activities, elevated serum levels of tumor necrosis factor (TNF)-alpha and beta, and detectable TNF-beta, interleukin (IL)-1beta and IL-6 mRNA in peripheral blood mononuclear cells (PBMC). CFS patients, as a group, also have significantly higher levels, as compared to controls, of soluble TNF receptor type I (sTNF-RI), sIL-6R and beta2-microglobulin (beta2-m), but not of IL-1 receptor antagonist (IL-1Ra). Correlative and population distribution studies that included lymphoid phenotypic distributions and function as well as soluble immune mediator expression levels revealed the existence of at least two mainly nonoverlapping categories among CFS patients with either: (1) dysregulated TNF—alpha/beta expression in association with changes in the serum levels of IL-1alpha, IL-4, sIL-2R, and IL-1Ra, PBMC-associated expression of IL-1beta, IL-6, and TNF-beta mRNA, and T cell activation; or (2) interrelated and dysregulated expression of sTNF-R1, sIL-6R, and beta2-microglobulin and significantly decreased lymphoproliferative and NK cell cytotoxic activities. Furthermore, allostasis—the ability to achieve stability through change—is critical to survival, and many psychological disorders are manifestations of the fact that such stability is not present. Through allostasis, the autonomic nervous system, the hypothalamic-pituitary-adrenal (HPA) axis, and the cardiovascular, metabolic, and immune systems protect the body by responding to internal and external stress. The price of this accommodation to stress can be allostatic load, which is the wear and tear that results from chronic overactivity or underactivity of allostatic systems.

The core of the body's response to a challenge is twofold, turning on an allostatic response that initiates a complex adaptive pathway, and then shutting off this response when the threat is past. The most common allostatic responses involve the sympathetic nervous systems and the HPA axis. For these systems, activation releases catecholamines from nerves and the adrenal medulla and leads to the secretion of corticotropin from the pituitary. The corticotropin, in turn, mediates the release of cortisol from the adrenal cortex. Inactivation returns the systems to base-line levels of cortisol and catecholamine secretion, which normally happens when the danger is past. However, if the inactivation is inefficient, there is overexposure to stress hormones.

Over weeks, months, or years, exposure to increased secretion of stress hormones results in a so-called allostatic load and its immunopathophysiologic consequences. It has been shown that allostatic load over a lifetime may cause the allostatic systems to wear out or become exhausted. Frailty in old age is generally seen as a consequence of a worn-out allostatic system. A vulnerable link in the regulation of the HPA axis and cognition is the hippocampal region. Wear and tear on this region of the brain leads to dysregulation of the HPA axis and cognitive impairment. Indeed, some, but not all, of the aging people have impairment of episodic, declarative, and spatial memory and hyperactivity of the HPA axis, all of which can be traced to inflammatory hippocampal damage. Recent data show that similar events occur at a younger age in humans with unexplained mood disorders. In one type of allostatic load inadequate responses by some allostatic systems trigger compensatory increases in others. When one system does not respond adequately to a stressful stimulus, the activity of other systems increases, because the underactive system is not providing the usual counter-regulation. For example, if cortisol secretion does not increase in response to stress, secretion of inflammatory cytokines (which are counter-regulated by cortisol) increases. The negative consequences of an enhanced inflammatory response are, for example, that the affected subjects are very susceptible to autoimmune and inflammatory disturbances, aggravated often by a genetically determined hyporesponsiveness of the HPA axis.

Also, the months following childbirth are a time when some women are susceptible to serious mood disorders. The illnesses can be resistant to conventional psychiatric treatment methods. Cases of postpartum depression or puerperal psychosis often occur in women with a past history of major depression or bipolar disorder. There has been considerable debate as to whether postpartum psychosis is a discrete diagnostic entity or whether it represents a rapidly evolving psychosis, which is a manifestation of an underlying bipolar (or manic-depressive) disorder. To date, existing psychiatric research supports the latter view.

Provided are methods for treating a subject believed to be suffering from a neurologic disorder, with a specific aim of reducing the frequency, and limit the lasting effects of the psychological manifestations of neuroimmune disease, and in particular the treatment of the inflammatory component of neurological or mood disorders to relieve symptoms that arise from the release of additional pro-inflammatory cytokines, in particular during disease progression, to prevent disability arising from disease progression, and to promote CNS tissue repair.

Provided is a pharmaceutical composition for the treatment of a neurological disorder occurring in a subject, for example in a primate, and a method for the treatment of the disease associated with additional pro-inflammatory cytokine release, for example in a primate comprising subjecting the subject to a signaling molecule according to the invention, preferably to a mixture of such signaling molecules.

The invention aims at countering the involvement of cell-mediated immunity in the etiology of neurologic disease, and treating the inflammatory component of neurological disorders by targeting the central role of NFkappaB-induced cytokine expression. As a consequence of (likely CNS-based) NFkappaB expression, toxic inflammatory mediators are released, sustaining breakdown of the blood-brain barrier and leading to injury of axons and glia. Nitric oxide might act directly on normal or hypomyelinated axons, transiently blocking conduction and reversibly increasing deficits arising from already compromised pathways. As acute inflammation resolves, pathways are released from nitric oxide-induced physiological conduction block. Symptoms also improve as surviving functional pathways are reorganized at the cellular and systems level. Together, these mechanisms account for remission early in the disease. But tissue vulnerability is easily exposed. When compounded by high axonal firing frequency, nitric oxide causes structural (and hence irreversible) changes to axons. Cytokines and growth-promoting factors released by reactive astrocytes and microglia as part of the acute inflammatory process promote endogenous remyelination. But, over time, astrocyte reactivity seals the lesion and gliosis causes a physical barrier to further remyelination, reducing the capacity to accommodate cumulative deficits, and marking transition to the stage of persistent deficit.

Since permanent disability can be caused by incomplete recovery from inflammation, provided is a method for modulating a neurological disorder in a subject believed to be in need thereof comprising providing the subject with a signaling molecule comprising a short, gene regulatory peptide or functional analogue thereof, wherein the signaling molecule is administered in an amount sufficient to modulate the exacerbating event. The signal molecule may be a short peptide, preferably at most 30 amino acids long, or a functional analogue or derivative thereof.

In certain embodiments, the peptide is an oligopeptide of from about three to about 15 amino acids long, preferably four to twelve, more preferably four to nine, most preferably four to six amino acids long, or a functional analogue or derivative thereof. Such a signaling molecule can be longer, for example, by extending it (N- and/or C-terminally), with more amino acids or other side groups, which can for example be (enzymatically) cleaved off when the molecule enters the place of final destination, however, by virtue of its small size of smaller than 15, preferably smaller than nine amino acids, a peptide or functional analogue according to the invention thereof readily crossing the blood brain barrier. Furthermore such a small peptide as provided herein is very stable and has a pharmaceutical half life greater than four hours.

Herewith, also provided is a method of treatment of mood disorders such as cases of postpartum depression or puerperal psychosis and a use of a signal molecule according to the invention for the preparation of a pharmaceutical composition for the treatment of cases of postpartum depression or puerperal psychosis, in particular by at least partly restoring or mimicking the anti-inflammatory activity of the gene-regulatory peptides LQGV (SEQ ID NO:1) and VLPALP (SEQ ID NO:4) and their functional analogues. In particular, a method is provided wherein the signaling molecule modulates translocation and/or activity of a gene transcription factor. It is particularly useful when the gene transcription factor comprises an NF-kappaB/Rel protein or an AP-1 protein. Many of the neurological disorders events as mentioned above involve increased expression of inflammatory cytokines due to activation of NF-κB and AP-1, and in certain embodiments provided is a method wherein translocation and/or activity of the NF-kappaB/Rel protein or AP-1 protein is inhibited. In this way, the destruction of brain tissues like the myelin lining of nerves or plaque formation which disrupts the brain which have been found to be significantly based on autoimmune or proinflammatory destruction caused by a dysregulated release of cytokines and chemokines is inhibited by a treatment according to the invention.

In one embodiment, the peptide is selected from the group of peptides LQG, AQG, LQGV (SEQ ID NO:1), AQGV (SEQ ID NO:2), LQGA (SEQ ID NO:3), VLPALP (SEQ ID NO:4), ALPALP (SEQ ID NO:5), VAPALP (SEQ ID NO:6), ALPALPQ (SEQ ID NO:7), VLPAAPQ (SEQ ID NO:8), VLPALAQ (SEQ ID NO:9), LAGV (SEQ ID NO:10), VLAALP (SEQ ID NO:11), VLAALP (SEQ ID NO:11), VLPALA (SEQ ID NO:12), VLPALPQ (SEQ ID NO:13), VLAALPQ (SEQ ID NO:14), VLPALPA (SEQ ID NO:15), GVLPALP (SEQ ID NO:16), LQGVLPALPQVVC (SEQ ID NO:17), LPGCPRGVNPVVS (SEQ ID NO:18), LPGC (SEQ ID NO:19), MTRV (SEQ ID NO:20), MTR, VVC. Preferred compounds are LQGV (SEQ ID NO:1), MTR, MTRV (SEQ ID NO:20), AQGV (SEQ ID NO:2), LAGV (SEQ ID NO:10), AQG, LQG, VLPALPQ (SEQ ID NO:13), LAG, and/or VLPALP. In particular, provided is a peptide for the treatment of a subject suffering from acute local brain inflammations (such as acute exacerbations believed to be due to MS) that may be selected from the group of LQGV (SEQ ID NO:1), MTR, MTRV (SEQ ID NO:20), AQGV (SEQ ID NO:2), LAGV (SEQ ID NO:10), AQG, and LQG. For acute systemic brain inflammations such as seen with stroke or another major ischemic event in the brain the peptide may be selected from the group of LAGV (SEQ ID NO:10), AQGV (SEQ ID NO:2) and LQGV (SEQ ID NO:1).

More in particular, provided is a peptide for the treatment of a subject suffering from acute exacerbations believed to be due to MS, selected from the group of LQGV (SEQ ID NO:1), MTR, and MTRV. Most in particular, provided is a pharmaceutical composition for the treatment of a subject suffering from acute exacerbations believed to be due to MS, the pharmaceutical composition comprising a pharmacologically effective amount of LQGV (SEQ ID NO:1) together with a pharmaceutically acceptable diluent. As said, additional expression of inflammatory cytokines is often due to activation of NF-κB and AP-1. Inflammatory cytokines can be expressed by endothelium, perivascular cells and adherent or transmigrating leukocytes, all inducing numerous pro-inflammatory and procoagulant effects. Together these effects predispose to inflammation, thrombosis and hemorrhage. Of clinical and medical interest and value, the present invention provides the opportunity to selectively control NFκB-dependent gene expression in tissues and organs in a living subject, preferably in a primate, allowing up-regulating essentially anti-inflammatory responses such as IL-10, and down-regulating essentially pro-inflammatory responses such as mediated by TNF-alpha, nitric oxide (NO), IL-5, IL-6 and IL-1beta.

The invention further provides a pharmaceutical composition for the treatment of a subject suffering from inflammatory neurological disorders the pharmaceutical composition comprising a pharmacologically effective amount of LQGV (SEQ ID NO:1), MTR, MTRV (SEQ ID NO:20), AQGV (SEQ ID NO:2), LAGV (SEQ ID NO:10), AQG, LQG, VLPALPQ (SEQ ID NO:13), LAG, and/or VLPALP (SEQ ID NO:4) together with a pharmaceutically acceptable diluent. In particular, provided is a pharmaceutical composition for the treatment of a subject suffering from inflammatory neurological disorders, the pharmaceutical composition comprising a pharmacologically effective amount of LQGV (SEQ ID NO:1), MTR, MTRV (SEQ ID NO:20), AQGV (SEQ ID NO:2), LAGV (SEQ ID NO:10), AQG, and/or LQG together with a pharmaceutically acceptable diluent.

More in particular, provided is a pharmaceutical composition for the treatment of a subject suffering from acute local brain inflammations, the pharmaceutical composition comprising a pharmacologically effective amount of LQGV (SEQ ID NO:1), MTR, and/or MTRV (SEQ ID NO:20) together with a pharmaceutically acceptable diluent.

Most in particular, provided is a pharmaceutical composition for the treatment of a subject suffering from acute exacerbations believed to be due to MS, the pharmaceutical composition comprising a pharmacologically effective amount of LQGV (SEQ ID NO:1) together with a pharmaceutically acceptable diluent.

A particularly useful pharmaceutically acceptable diluent is sterile water or an isotonic salt solution such as 0.9% saline or phosphate buffered salt (PBS). The peptide can be administered and introduced in-vivo preferably via any route, and via passage through the mucosae or skin. The peptide, or its modification or derivative, can be administered as the entity as such or as a pharmaceutically acceptable acid- or base-addition salt, formed by reaction with an inorganic acid (such as hydrochloric acid, hydrobromic acid, perchloric acid, nitric acid, thiocyanic acid, sulfuric acid, and phosphoric acid); or with an organic acid (such as formic acid, acetic acid, propionic acid, glycolic acid, lactic acid, pyruvic acid, oxalic acid, malonic acid, succinic acid, maleic acid, and fumaric acid); or by reaction with an inorganic base (such as sodium hydroxide, ammonium hydroxide, potassium hydroxide); or with an organic base (such as mono-, di-, trialkyl and aryl amines and substituted ethanolamines). A selected peptide and any of the derived entities may also be conjugated to DMSO, translocating peptides, sugars, lipids, other polypeptides, nucleic acids and PNA; and function in-situ as a conjugate or be released locally after reaching a targeted tissue or organ. Herein, provided is a further selection of compounds useful for the treatment of inflammatory neurological disorders and other diseases such as atherosclerosis wherein foamy cells may involved in the disease etiology. Preferred compounds are LQGV (SEQ ID NO:1), MTR, MTRV (SEQ ID NO:20), AQGV (SEQ ID NO:2), LAGV (SEQ ID NO:10), AQG, LQG, VLPALPQ (SEQ ID NO:13), LAG, and/or VLPALP. More preferred compounds are LQGV (SEQ ID NO:1), MTR, MTRV (SEQ ID NO:20), AQGV (SEQ ID NO:2), LAGV (SEQ ID NO:10), AQG, and LQG. Most preferred compounds are LQGV (SEQ ID NO:1), MTR, and MTRV. Single most preferred compound is LQGV (SEQ ID NO:1). Doses of 1 to 5 mg/kg bodyweight, for example every eight hours in a bolus injection or per infusionem until the patient stabilizes, are recommended initially, however, the potential of oral treatment allows a rapid transition to oral administration thereafter. For example in cases where large adverse response are expected or diagnosed, it is preferred to monitor cytokine profiles, such as TNF-alpha, IL-6 or IL-10 levels, in the plasma of the treated patient, and to stop treatment according to the invention when these levels are normal.

In certain embodiments, it is herein provided to give a patient experiencing a severe and acute stroke or major cerebrovascular event with a bolus injection LQGV (SEQ ID NO:1), AQGV (SEQ ID NO:2) or LAGV (SEQ ID NO:10) or other peptide as provided herein at 0.1 to 30 mg/kg, preferably at 1 to 10 mg/kg, such as at 2 mg/kg and continue the infusion with LQGV (SEQ ID NO:1), AQGV (SEQ ID NO:2), LAGV (SEQ ID NO:10) or another preferred peptide at a dose of 1 mg/kg bodyweight for every eight hours. The oral treatment commences, using dosages of 0.01 to 10 mg/kg bodyweight, and preferably 0.1 to 1 mg/kg bodyweight until the cerebrovascular event has stabilized. Dosages may be increased or decreased, for example depending on the outcome of monitoring the cytokine profile in the plasma or CSF of the patient. Although the peptide may be prepared by other methods known for the preparation of analogous compounds (e.g., by use of a solid phase synthesis), a method of making the peptide is described in the detailed description herein. During the process of preparation, solvents such as N, N-dimethylformamide (DMF), 1-butanol, 2-butanol, ethanol, methanol, ethyl acetate, methylene chloride, hexane, diethyl ether, water, acetic acid, and others may be used. Catalysts containing palladium or molybdenum may also be used in the preparation of the peptide.

However made, the peptide forms pharmacologically acceptable salts from pharmacologically acceptable organic and inorganic acids such as hydrochloric, hydrobromic, fumaric, phosphoric, ascorbic, tartaric, citric, lactic, maleic, palmitic, and other well-known acids. Especially preferred are the hydrochloric and acetic acid salts. The acid addition salts are obtained by reacting the peptide with the acid.

Methods of crystallizing compounds are described in Chase et al., Remington's Pharmaceutical Sciences (16th ed., Mack Publishing Co., Easton. PA, U.S.A., 1980) (“Remington's”), at page 1535.

A crystalline peptide can be used to make numerous dosage forms such as powders for insufflations, powders for reconstitution, tablet triturates (e.g., dispensing tablets and hypodermic tablets), other tablets, and so forth.

The pharmaceutical compositions containing the crystalline peptide are preferably dispensed in unit dosage forms, such as tablets, capsules, pills, powders, granules, suppositories, sterile parenteral solutions or suspensions and non-parenteral solutions or suspensions, containing suitable quantities of the pharmaceutically acceptable salt of the peptide. Methods and compositions for making such dosage forms are well-known to those skilled in the art. For example, methods of making powders and their compositions are described at pages 1535 through 1552 of Remington's. Insufflations are described at page 1552, and insufflators are described at 1792. Methods and compositions for making tablets and pills, containing active ingredients, are described in Remington's, at pages 1553 through 1584. Methods of coating pharmaceutical dosage forms and making prolonged release pharmaceuticals are described at pages 1585-1613 of Remington's. The contents of these pages are hereby incorporated by this reference.

The crystalline peptide may also be incorporated into devices intended for implantation into a patient. Such devices, polymers intended for use therein, and methods of making both are described in U.S. Pat. Nos. 3,773,919, 4,767,628, and 4,675,189. For example, a sufficient quantity of the crystalline peptide could be incorporated into a PLAGA implant to allow for the release of peptide (e.g., 5 mg per day for one month) into the patient's body.

One advantage with pharmaceutical compositions containing the crystalline versus the amorphous product, is that the pharmaceutical composition containing the crystalline salt product, having twice the bioavailability of the amorphous product, may need only contain half the absolute amount of the active ingredient on certain mucosa thus decreasing the amount of ingredient needed to be insufflated or otherwise administered and decreasing the ultimate cost of the composition. Such mucosa would include the nasal and the buccal mucosa.

Although the pharmaceutical compositions containing the crystalline peptide may be formulated with adjuvants such as solubilizers, they need not be. The ability to use solely the crystalline peptide (i.e., the crystalline acid addition salt of the peptide) in a pharmaceutical composition to be applied to, for example, a nasal mucosa has advantages. For one thing, certain adjuvants are not suitable for chronic administration. However, long term administration may be necessary for the particular person ingesting the peptide. Another advantage is that the adjuvants necessarily take up a portion of the pharmaceutical composition, which portion may be better suited for the peptide in order to decrease mucosal discomfort.

However if it is desired, suitable solubilizers, buffers, swelling agents, etc., may be used in such formulations. Buffering agents are preferably those which keep the peptide in its unionized form.

The dosage of the crystalline acid addition salt/peptide administered will generally be dependent upon the kind of disorder to be treated, the type of patient involved, his age, health, weight, kind of concurrent treatment, if any, and length and frequency of treatment.

The dosage forms will be administered over varying durations. To treat a disorder, the compounds are administered to a patient for a length of time sufficient to alleviate the symptoms associated with the disorders that the patient is suffering from. This time will vary, but periods of time exceeding two months are especially preferred. After the symptoms have been alleviated, the compound may then be discontinued to determine whether it is still required by the particular patient.

### EXAMPLES

The invention thus provides use of a NFκB regulating peptide or derivative thereof for the production of a pharmaceutical composition for the treatment of inflammatory neurological disorders, preferably in a primate, and provides a method of treatment of neurological disorders, notably in a primate. It is preferred when the treatment comprises administering to the subject a pharmaceutical composition comprising an NFkappaB down-regulating peptide or functional analogue thereof. Examples of useful NFkappaB down-regulating peptides are VLPALPQVVC, LQGVLPALPQ, LQG, LQGV (SEQ ID NO:1), GVLPALPQ, VLPALP (SEQ ID NO:4), VVC, MTR and circular LQGVLPALPQVVC. More down-regulating peptides and functional analogues can be found using the methods as provided herein. Most prominent among NFkappaB down-regulating peptides are VLPALPQVVC, LQGVLPALPQ, LQG, LQGV (SEQ ID NO:1), and VLPALP. These are also capable of reducing production of NO by a cell. It is herein also provided to use a composition that comprises at least two oligopeptides or functional analogues thereof, each capable of down-regulation NFkappaB, and thereby reducing production of NO and/or TNF-alpha by a cell, in particular wherein the at least two oligopeptides are selected from the group LQGV (SEQ ID NO:1), AQGV (SEQ ID NO:2) and VLPALP (SEQ ID NO:4), for the treatment of recurring disease seen with neurological disorders.

In response to a variety of signals received by the body in the course of the disease, the NFB/Rel family of transcription factors are activated and form different types of hetero- and homodimers among themselves to regulate the expression of target genes containing kappaB-specific binding sites. NF-kB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFkB is a heterodimer of p65 (RELA) and p50 (NF-kB1). Among the activated NFkB dimers, p50-p65 heterodimers are known to be involved in enhancing the transcription of target genes and p50-p50 homodimers in transcriptional repression. However, p65-p65 homodimers are known for both transcriptional activation and repressive activity against target genes. KappaB DNA binding sites with varied affinities to different NFB dimers have been discovered in the promoters of several eukaryotic genes and the balance between activated NFkB homo- and heterodimers ultimately determines the nature and level of gene expression within the cell.

The term “NFkB-regulating peptide” as used herein refers to a peptide or a modification or derivative thereof capable of modulating the activation of members of the NFkB/Rel family of transcription factors. Activation of NFkB can lead to enhanced transcription of target genes. Also, it can lead to transcriptional repression of target genes. NFkB activation can be regulated at multiple levels. For example, the dynamic shuttling of the inactive NFkB dimers between the cytoplasm and nucleus by IkappaB proteins and its termination by phosphorylation and proteasomal degradation, direct phosphorylation, acetylation of NFkB factors, and dynamic reorganization of NFkB subunits among the activated NFkB dimers have all been identified as key regulatory steps in NFkB activation and, consequently, in NFkB-mediated transcription processes. Thus, an NFkB-regulating peptide is capable of modulating the transcription of pro-inflammatory cytokine genes that are under the control of NFkB/Rel family of transcription factors. Modulating comprises the up-regulation or the downregulation of transcription.

In certain embodiments, a peptide according to the invention, or a functional derivative or analogue thereof is used for the production of a pharmaceutical composition for the treatment of neurological disorders. NFkappaB regulating peptide can be given alone or concomitantly to other treatments, the peptide (or analogue) concentration preferably being from about 1 to about 1000 mg/l, but the peptide can also been given on its own, for example in a bolus injection. In acute cases, doses of 1 to 5 mg/kg bodyweight, for example every eight hours in a bolus injection or per infusionem until the patient stabilizes, are recommended. For example in cases where large adverse response are expected or diagnosed, it is preferred to monitor cytokine profiles, such as TNF-alpha, IL-6 or IL-10 levels, in the plasma of the treated patient, and to stop treatment according to the invention when these levels are normal.

In certain embodiments, it is herein provided to provide the patient experiencing a severe and acute bipolar disorder with a bolus injection of NF-kappaB down-regulating peptide such as AQGV (SEQ ID NO:2), LQGV (SEQ ID NO:1) or VLPALP (SEQ ID NO:4) at 2 mg/kg and continue the infusion with a NF-kappaB down regulating peptide such as AQGV (SEQ ID NO:2), LQGV (SEQ ID NO:1) or VLPALP (SEQ ID NO:4) or a functional analogue thereof at a dose of 1 mg/kg bodyweight for every eight hours. Dosages may be increased or decreased, for example depending on the outcome of monitoring the cytokine profile in the plasma or CSF of the patient. As said, disease progression is dramatically mediated by cytokines and chemokines. For example, the TNF-alpha family is then highly elevated in CSF. The down-regulation or T cell regulation of these cytokines and chemokines can prevent T cell and dendritic cells from reaching the CNS and then further down-regulate the proinflammatory response which produces pathology of the brain and spinal cord. This model of migration of cells to the CNS and then the release of proinflammatory cytokines and chemokines is seen in the following and can be treated by a peptide according to the invention through NFkappB regulation, the development of T regulator cells, and the intervention of expression early or pregenes such as C-jun or C-erg. For the pathologist, neurological disorders often present as a disorder of the central nervous system, manifesting as acute focal inflammatory demyelination and axonal loss with limited remyelination. Thus, the primary nature of inflammation is undisputed and is central for treatments that modulate the immune system. There are, however, several aspects that limit the therapeutic efficacy of strategies directed against the inflammatory component of the disease. Currently, immune suppression with corticosteroids is unable to specifically stop the inflammatory regimes. Also, the inflammatory forms of neurological disorder, such as described above with autism, which are now epidemic in US and European studies responds well in part to the use of a NFkappaB down regulating peptides according to the invention.

In response to a variety of pathophysiological and developmental signals, the NFkB/Rel family of transcription factors are activated and form different types of hetero- and homodimers among themselves to regulate the expression of target genes containing kappaB-specific binding sites. NF-kB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFkB is a heterodimer of p65 (RELA) and p50 (NF-kB1). Among the activated NFkB dimers, p50-p65 heterodimers are known to be involved in enhancing the transcription of target genes and p50-p50 homodimers in transcriptional repression. However, p65-p65 homodimers are known for both transcriptional activation and repressive activity against target genes. KappaB DNA binding sites with varied affinities to different NFB dimers have been discovered in the promoters of several eukaryotic genes and the balance between activated NFkB homo- and heterodimers ultimately determines the nature and level of gene expression within the cell.

The term “NFkB-regulating peptide” as used herein refers to a peptide or a modification or derivative thereof capable of modulating the activation of members of the NFkB/Rel family of transcription factors. Activation of NFkB can lead to enhanced transcription of target genes. Also, it can lead to transcriptional repression of target genes. NFkB activation can be regulated at multiple levels. For example, the dynamic shuttling of the inactive NFkB dimers between the cytoplasm and nucleus by IkappaB proteins and its termination by phosphorylation and proteasomal degradation, direct phosphorylation, acetylation of NFkB factors, and dynamic reorganization of NFkB subunits among the activated NFkB dimers have all been identified as key regulatory steps in, NFkB activation and, consequently, in NFkB-mediated transcription processes. Thus, an NFkB-regulating peptide is capable of modulating the transcription of genes that are under the control of NFkB/Rel family of transcription factors. Modulating comprises the up-regulation or the down-regulation of transcription. In certain embodiments, a peptide according to the invention, or a functional derivative or analogue thereof is used for the production of a pharmaceutical composition. Examples of useful NFkappaB down-regulating peptides to be included in such a pharmaceutical composition are VLPALPQVVC, LQGVLPALPQ, LQG, LQGV (SEQ ID NO:1), GVLPALPQ, VLPALP (SEQ ID NO:4), VVC, MTR and circular LQGVLPALPQVVC. More gene-regulating peptides and functional analogues can be found in a (bio)assay, such as a NFkappaB translocation assay as provided herein. Most prominent among NFkappaB down-regulating peptides are VLPALPQVVC, LQGVLPALPQ, LQG, LQGV (SEQ ID NO:1), and VLPALP. These are also capable of reducing production of NO by a cell. Furthermore, LQG, VVC and MTRV (SEQ ID NO:20), and in particular LQGV (SEQ ID NO:1) promote angiogenesis, especially in topical applications.

It is herein also provided to use a composition that comprises at least two oligopeptides or functional analogues thereof, each capable of down-regulation NFkappaB, and thereby reducing production of NO and/or TNF-alpha by a cell, in particular wherein the at least two oligopeptides are selected from the group LQGV (SEQ ID NO:1), AQGV (SEQ ID NO:2) and VLPALP. Useful NFkappaB up-regulating peptides are VLPALPQ (SEQ ID NO:13), GVLPALP and MTRV. As indicated, more gene-regulatory peptides may be found with an appropriate (bio)assay. A gene-regulatory peptide as used herein may be short. Preferably, such a peptide is three to 15 amino acids long, more preferably, wherein the lead peptide is three to nine amino acids long, most preferred wherein the lead peptide is four to six amino acids long, and capable of modulating the expression of a gene, such as a cytokine, in a cell. In certain embodiments, a peptide is a signaling molecule that is capable of traversing the plasma membrane of a cell or, in other words, a peptide that is membrane-permeable.

Functional derivative or analogue herein relates to the signaling molecular effect or activity as for example can be measured by measuring nuclear translocation of a relevant transcription factor, such as NF-kappaB in an NF-kappaB assay, or AP-1 in an AP-1 assay, or by another method as provided herein. Fragments can be somewhat (i.e., one or two amino acids) smaller or larger on one or both sides, while still providing functional activity. Such a bioassay comprises an assay for obtaining information about the capacity or tendency of a peptide, or a modification thereof, to regulate expression of a gene. A scan with for example a 15-mer, or a 12-mer, or a 9-mer, or a 8-mer, or a 7-mer, or a 6-mer, or a 5-mer, or a 4-mer or a 3-mer peptides can yield valuable information on the linear stretch of amino acids that form an interaction site and allows identification of gene-regulatory peptides that have the capacity or tendency to regulate gene expression. Gene-regulatory peptides can be modified to modulate their capacity or tendency to regulate gene expression, which can be easily assayed in an in vitro bioassay such as a reporter assay. For example, some amino acid at some position can be replaced with another amino acid of similar or different properties. Alanine (Ala)-replacement scanning, involving a systematic replacement of each amino acid by an Ala residue, is a suitable approach to modify the amino acid composition of a gene-regulatory peptide when in a search for a signaling molecule capable of modulating gene expression. Of course, such replacement scanning or mapping can be undertaken with amino acids other than Ala as well, for example with D-amino acids.

In one embodiment, a peptide derived from a naturally occurring polypeptide is identified as being capable of modulating gene expression of a gene in a cell. Subsequently, various synthetic Ala-mutants of this gene-regulatory peptide are produced. These Ala-mutants are screened for their enhanced or improved capacity to regulate expression of a gene compared to gene-regulatory polypeptide.

Furthermore, a gene-regulatory peptide, or a modification or analogue thereof, can be chemically synthesized using D- and/or L-stereoisomers. For example, a gene-regulatory peptide that is a retro-inverso of an oligopeptide of natural origin is produced. The concept of polypeptide retro-inversion (assemblage of a natural L-amino acid-containing parent sequence in reverse order using D-amino acids) has been applied successfully to synthetic peptides. Retro-inverso modification of peptide bonds has evolved into a widely used peptidomimetic approach for the design of novel bioactive molecules which has been applied to many families of biologically active peptide.

The sequence, amino acid composition and length of a peptide will influence whether correct assembly and purification are feasible. These factors also determine the solubility of the final product. The purity of a crude peptide typically decreases as the length increases. The yield of peptide for sequences less than 15 residues is usually satisfactory, and such peptides can typically be made without difficulty. The overall amino acid composition of a peptide is an important design variable. A peptide's solubility is strongly influenced by composition. Peptides with a high content of hydrophobic residues, such as Leu, Val, Ile, Met, Phe and Trp, will either have limited solubility in aqueous solution or be completely insoluble. Under these conditions, it can be difficult to use the peptide in experiments, and it may be difficult to purify the peptide if necessary. To achieve a good solubility, it is advisable to keep the hydrophobic amino acid content below 50% and to make sure that there is at least one charged residue for every five amino acids. At physiological pH Asp, Glu, Lys, and Arg all have charged side chains. A single conservative replacement, such as replacing Ala with Gly, or adding a set of polar residues to the N- or C-terminus, may also improve solubility.

Peptides containing multiple Cys, Met, or Trp residues can also be difficult to obtain in high purity partly because these residues are susceptible to oxidation and/or side reactions. If possible, one should choose sequences to minimize these residues. Alternatively, conservative replacements can be made for some residues. For instance, Norleucine can be used as a replacement for Met, and Ser is sometimes used as a less reactive replacement for Cys. If a number of sequential or overlapping peptides from a protein sequence are to be made, making a change in the starting point of each peptide may create a better balance between hydrophilic and hydrophobic residues. A change in the number of Cys, Met, and Trp residues contained in individual peptides may produce a similar effect.

In another embodiment of the invention, a gene-regulatory peptide capable of modulating gene expression is a chemically modified peptide. A peptide modification includes phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal succinylation, tripalmitoyl-S-Glyceryl Cysteine (PAM3 Cys-OH) as well as farnesylation of a Cys residue. Systematic chemical modification of a gene-regulatory peptide can, for example, be performed in the process of gene-regulatory peptide optimalization.

Synthetic peptides can be obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products.

The peptides as mentioned in this document such as LQG, AQG, LQGV (SEQ ID NO:1), AQGV (SEQ ID NO:2), LQGA (SEQ ID NO:3), VLPALP (SEQ ID NO:4), ALPALP (SEQ ID NO:5), VAPALP (SEQ ID NO:6), ALPALPQ (SEQ ID NO:7), VLPAAPQ (SEQ ID NO:8), VLPALAQ (SEQ ID NO:9), LAGV (SEQ ID NO:10), VLAALP (SEQ ID NO:11), VLPALA (SEQ ID NO:12), VLPALPQ (SEQ ID NO:13), VLAALPQ (SEQ ID NO:14), VLPALPA (SEQ ID NO:15), GVLPALP (SEQ ID NO:16), VVCNYRDVRFESIRLPGCPRGVNPVVSYAVALSCQCAL (SEQ ID NO: ______0, RPRCRPINATLAVEKEGCPVCITVNTTICAGYCPT (SEQ ID NO: ______), SKAPPPSLPSPSRLPGPS (SEQ ID NO: ______), LQGVLPALPQVVC (SEQ ID NO:17), SIRLPGCPRGVNPVVS, LPGCPRGVNPVVS (SEQ ID NO:18), LPGC (SEQ ID NO:19), MTRV (SEQ ID NO:20), MTR, and VVC were prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the alpha-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP.

Upon completion of the synthesis, the peptide resin was treated with a mixture of trifluoroacetic acid (TFA)/H₂O/triisopropylsilane (TIS) 95:2.5:2.5. After 30 minutes TIS was added until decolorization. The solution was evaporated in vacuo and the peptide precipitated with diethylether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/minute; absorbance was detected from 190-370 nm. There were different gradient systems used. For example for peptides LQG and LQGV: ten minutes 100% A followed by linear gradient 0-10% B in 50 minutes. For example for peptides VLPALP (SEQ ID NO:4) and VLPALPQ: five minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

RAW 264.7 macrophages, obtained from American Type Culture Collection (Manassas, Va.), were cultured at 37° C. in 5% CO₂ using DMEM containing 10% FBS and antibiotics (100 U/ml of penicillin, and 100 μg/ml streptomycin). Cells (1×10⁶/ml) were incubated with peptide (10 μg/ml) in a volume of 2 ml. After eight hours of cultures, cells were washed and prepared for nuclear extracts.

Nuclear extracts and EMSA were prepared according to Schreiber et al. Methods (Schreiber et al. 1989, Nucleic Acids Research 17). Briefly, nuclear extracts from peptide stimulated or nonstimulated macrophages were prepared by cell lysis followed by nuclear lysis. Cells were then suspended in 400 μl of buffer (10 mM HEPES (pH 7.9), 10 mM KCl, 0.1 mM KCL, 0.1 mM EDTA, 0.1 mM EGTA, 1 mM DTT, 0.5 mM PMSF and protease inhibitors), vigorously vortexed for 15 seconds, left standing at 4° C. for 15 minutes, and centrifuged at 15,000 rpm for two minutes. The pelleted nuclei were resuspended in buffer (20 mM HEPES (pH 7.9), 10% glycerol, 400 mM NaCl, 1 mM EDTA, 1 mM EGTA, 1 mM DTT, 0.5 mM PMSF and protease inhibitors) for 30 minutes on ice, then the lysates were centrifuged at 15,000 rpm for two minutes. The supernatants containing the solubilized nuclear proteins were stored at −70° C. until used for the Electrophoretic Mobility Shift Assays (EMSA).

Electrophoretic mobility shift assays were performed by incubating nuclear extracts prepared from control (RAW 264.7) and peptide treated RAW 264.7 cells with a 32P-labeled double-stranded probe (5′ AGCTCAGAGGGGGACTTTCCGAGAG 3′ (SEQ ID NO: ______) synthesized to represent the NF-kappaB binding sequence. Shortly, the probe was end-labeled with T4 polynucleotide kinase according to manufacturer's instructions (Promega, Madison, Wis.). The annealed probe was incubated with nuclear extract as follows: in EMSA, binding reaction mixtures (20 μl) contained 0.25 μg of poly(dI-dC) (Amersham Pharmacia Biotech) and 20,000 rpm of 32P-labeled DNA probe in binding buffer consisting of 5 mM EDTA, 20% Ficoll, 5 mM DTT, 300 mM KCl and 50 mM HEPES. The binding reaction was started by the addition of cell extracts (10 μg) and was continued for 30 minutes at room temperature. The DNA-protein complex was resolved from free oligonucleotide by electrophoresis in a 6% polyacrylamide gel. The gels were dried and exposed to x-ray films. The transcription factor NF-kB participates in the transcriptional regulation of a variety of genes. Nuclear protein extracts were prepared from LPS and peptide treated RAW264.7 cells or from LPS treated RAW264.7 cells. In order to determine whether the peptide modulates the translocation of NF-kB into the nucleus, on these extracts EMSA was performed. Here we see that indeed some peptides are able to modulate the translocation of NF-kB since the amount of labeled oligonucleotide for NF-kB is reduced. In this experiment peptides that show the modulation of translocation of NF-kB are: VLPALPQVVC (SEQ ID NO: ______), LQGVLPALPQ (SEQ ID NO: ______), LQG, LQGV (SEQ ID NO:1), GVLPALPQ (SEQ ID NO: ______), VLPALP (SEQ ID NO:4), VLPALPQ (SEQ ID NO:13), GVLPALP (SEQ ID NO:16), VVC, MTRV (SEQ ID NO:20), MTR.

RAW 264.7 mouse macrophages were cultured in DMEM, containing 10% or 2% FBS, penicillin, streptomycin and glutamine, at 37° C., 5% CO₂. Cells were seeded in a 12-well plate (3×10⁶ cells/ml) in a total volume of 1 ml for two hours and then stimulated with LPS (E. coli 026:B6; Difco Laboratories, Detroit, Mich., USA) and/or NMPF (1 microgr/ml). After 30 minutes of incubation, plates were centrifuged and cells were collected for nuclear extracts. Nuclear extracts and EMSA were prepared according to Schreiber et al. Cells were collected in a tube and centrifuged for five minutes at 2000 rpm (rounds per minute) at 4° C. (Universal 30 RF, Hettich Zentrifuges). The pellet was washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five microliters 10% NP-40 was added and the sample was centrifuged (two minutes, 4000 rpm, 4° C.). The supernatant (cytoplasmic fraction) was collected and stored at −70° C. The pellet, which contains the nuclei, was washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples were left to shake at 4° C. for at least 60 minutes. Finally the samples were centrifuged and the supernatant (nucleic fraction) was stored at −70° C.

Bradford reagent (Sigma) was used to determine the final protein concentration in the extracts. For Electrophoretic mobility shift assays an oligonucleotide representing NF-κB binding sequence (5′-AGC TCA GAG GGG GAC TTT CCG AGA G-3′ (SEQ ID NO: ______)) was synthesized. Hundred pico mol sense and antisense oligo were annealed and labeled with γ-³²P-dATP using T4 polynucleotide kinase according to manufacturer's instructions (Promega, Madison, Wis.). Nuclear extract (5-7.5 μg) was incubated for 30 minutes with 75000 cpm probe in binding reaction mixture (20 microliters) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. The DNA-protein complex was resolved from free oligonucleotide by electrophoresis in a 4-6% polyacrylamide gel (150 V, two to four hours). The gel was then dried and exposed to x-ray film. The transcription factor NF-κB participates in the transcriptional regulation of a variety of genes. Nuclear protein extracts were prepared from either LPS (1 mg/ml), peptide (1 mg/ml) or LPS in combination with peptide treated and untreated RAW264.7 cells. In order to determine whether the peptides modulate the translocation of NF-κB into the nucleus, on these extracts EMSA was performed. Peptide signaling molecules are able to modulate the basal as well as LPS induced levels of NF-κB. In this experiment peptides that show the inhibition of LPS induced translocation of NF-kB are: VLPALPQVVC (SEQ ID NO: ______), LQGVLPALPQ (SEQ ID NO: ______), LQG, LQGV (SEQ ID NO:1), GVLPALPQ, VLPALP (SEQ ID NO:4), VVC, MTR and circular LQGVLPALPQVVC (SEQ ID NO:17). Peptide signaling molecules that in this experiment promote LPS induced translocation of NF-κB are: VLPALPQ (SEQ ID NO:13), GVLPALP (SEQ ID NO:16), and MTRV (SEQ ID NO:20). Basal levels of NF-κB in the nucleus was decreased by VLPALPQVVC, LQGVLPALPQ, LQG and LQGV (SEQ ID NO:1) while basal levels of NF-κB in the nucleus was increased by GVLPALPQ (SEQ ID NO: ______), VLPALPQ (SEQ ID NO:13), GVLPALP (SEQ ID NO:16), VVC, MTRV (SEQ ID NO:20), MTR and LQGVLPALPQVVC (SEQ ID NO:17). In other experiments, QVVC (SEQ ID NO: ______) also showed the modulation of translocation of NF-κB into nucleus (data not shown).

### Further Modes of Identification of Gene-Regulatory Peptides by NF-κB Analysis

Cells: Cells will be cultured in appropriate culture medium at 37° C., 5% CO₂. Cells will be seeded in a 12-well plate (usually 1×10⁶ cells/ml) in a total volume of 1 ml for two hours and then stimulated with regulatory peptide in the presence or absence of additional stimuli such as LPS. After 30 minutes of incubation plates will be centrifuged and cells are collected for cytosolic or nuclear extracts.

Nuclear Extracts Nuclear extracts and EMSA could be prepared according to Schreiber et al. Method (Schriber et al. 1989, Nucleic Acids Research 17). Cells are collected in a tube and centrifuged for five minutes at 2000 rpm (rounds per minute) at 4° C. (Universal 30 RF, Hettich Zentrifuges). The pellet is washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five microliters 10% NP-40 is added and the sample is centrifuged (two minutes, 4000 rpm, 4° C.). The supernatant (cytoplasmic fraction) was collected and stored at −70° C. for analysis. The pellet, which contains the nuclei, is washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples are left to shake at 4° C. for at least 60 minutes. Finally the samples are centrifuged and the supernatant (nucleic fraction) is stored at −70° C. for analysis. Bradford reagent (Sigma) could be used to determine the final protein concentration in the extracts.

EMSA: For Electrophoretic mobility shift assays an oligonucleotide representing NF-κB binding sequence such as (5′-AGC TCA GAG GGG GAC TTT CCG AGA G-3′ (SEQ ID NO: ______)) are synthesized. Hundred pico mol sense and antisense oligo are annealed and labeled with γ-³²P-dATP using T4 polynucleotide kinase according to manufacturer's instructions (Promega, Madison, Wis.). Cytosolic extract or nuclear extract (5-7.5 μg) from cells treated with regulatory peptide or from untreated cells is incubated for 30 minutes with 75000 cpm probe in binding reaction mixture (20 μl) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. Or cytosolic and nuclear extract from untreated cells or from cells treated with stimuli could also be incubated with probe in binding reaction mixture and binding buffer. The DNA-protein complex are resolved from free oligonucleotide by electrophoresis in a 4-6% polyacrylamide gel (150 V, two to four hours). The gel is then dried and exposed to x-ray film. Peptides can be biotinylated and incubated with cells. Cells are then washed with phosphate-buffered saline, harvested in the absence or presence of certain stimulus (LPS, PHA, TPA, anti-CD3, VEGF, TSST-1, VIP or know drugs etc.). After culturing cells are lysed and cells lysates (whole lysate, cytosolic fraction or nuclear fraction) containing 200 micro gram of protein are incubated with 50 microliters Neutr-Avidin-plus beads for one hour at 4° C. with constant shaking. Beads are washed five times with lysis buffer by centrifugation at 6000 rpm for one minute. Proteins are eluted by incubating the beads in 0.05 N NaOH for one minute at room temperature to hydrolyze the protein-peptide linkage and analyzed by SDS-polyacrylamide gel electrophoresis followed by immunoprecipitated with agarose-conjugated anti-NF-κB subunits antibody or immunoprecipitated with antibody against to be studied target. After hydrolyzing the protein-peptide linkage, the sample could be analyzed on HPLS and mass-spectrometry. Purified NF-κB subunits or cell lysate interaction with biotinylated regulatory peptide can be analyzed on biosensor technology. Peptides can be labeled with FITC and incubated with cells in the absence or presence of different stimulus. After culturing, cells can be analyzed with fluorescent microscopy, confocal microscopy, flow cytometry (cell membrane staining and/or intracellular staining) or cells lysates are made and analyzed on HPLC and mass-spectrometry. NF-κB transfected (reporter gene assay) cells and gene array technology can be used to determine the regulatory effects of peptides.

HPLC and mass-spectrometry analysis: Purified NF-κB subunit or cytosolic/nuclear extract is incubated in the absence or presence of (regulatory) peptide is diluted (2:1) with 8 N guanidinium chloride and 0.1% trifluoroacetic acid, injected into a reverse-phase HPLC column (Vydac C18) equilibrated with solvent A (0.1% trifluoroacetic acid), and eluted with a gradient of 0 to 100% eluant B (90% acetonitrile in solvent A). Factions containing NF-kB subunit are pooled and concentrated. Fractions are then dissolved in appropriate volume and could be analyzed on mass-spectrometry.

Compounds were further selected in a method for assessing or determining activity of a test compound on modulation of gene product levels comprising culturing (preferably myeloid) cells, contacting at least one of the cultured cells with a lipid-rich fraction, contacting at least one of the cultured cells with the test compound, determining the presence of a gene product of at least one cell of the cultured cells, and optionally determining the presence of the gene product of at least one cultured cell not contacted with the test compound. To assess human conditions most fully, it is preferred that the cell is of human origin, for example a peripheral blood monocyte or granulocyte taken from a healthy donor. Also, provided is a method for assessing or determining activity of a test compound on modulation of gene product levels in more specific circumstances of disease, it is then preferred the myeloid cell has been derived from a subject thought to be suffering from a disease. Use of a method according to the invention would then allow for individualized medicine; test results indicating that a specific test compound has specific benefits for the subject may then be used for treatment of the subject against the disease.

In particular, provided is a method to practice an in vitro model of brain inflammation such as MS. This method for example comprises a step of culturing a (preferably myeloid) cell or cells, preferably of human origin, such as a human blood monocyte obtained from a donor, if required differentiating the monocyte into other cell types such as macrophages and dendritic cells and a step of contacting the cultured cell with a lipid-rich fraction, preferably a phospholipid rich fraction, preferably with a myelin-rich fraction and a third step of culturing the cell in the presence of the lipid-rich fraction until the cell or at least 10% of the cells, preferably at least 20%, more preferably at least 30%, more preferably at least 30%, more preferably at least 40%, more preferably at least 50%, more preferably at least 60%, more preferably at least 70%, more preferably at least 80%, most preferably at least 90%, have developed a foamy characteristic because of the ingestion of the lipid-rich fraction, as can be observed by light microscope or as can be determined by staining the cell or cells for the intracellular presence of lipid-rich fractions, as for example can be done by staining the cell or cells or a fraction thereof with a stain for the detection of neutral lipids, such as by staining with oil red O histochemistry (ORO) or by fluorescent labeling of lipids with DiI and subsequent detection of ingested fluorescent lipids. Letting foam cells stand in culture for a too long period without feeding a lipid-rich fraction will make them return to a non-foamy character; it then suffices to re-feed them a lipid-rich fraction to induce the foamy morphology again. In one embodiment of the invention, human myeloid cells obtained from healthy donors are fed with 10 to 200, preferably with about 50 microg/ml human myelin for example purified from postmortem brain.

In another embodiment, mouse primary macrophages obtained from healthy mice are fed with 10 to 200, preferably with about 50 microg/ml human or mouse myelin. In another embodiment of the invention, marmoset myeloid cells obtained from healthy donors are fed with 10 to 200, preferably with about 50 microg/ml marmoset myelin. In another embodiment of the invention, human primary macrophages obtained from healthy donors are fed with 10 to 200, preferably with about 50 microg/ml phospholipid. Although small individual changes in kinetics between individual donors may be observed, myeloid cells acquire a foamy morphology between 24 and 48 hours and contain a markedly increased number and size of lipid droplets in comparison to control cells (i.e., not fed with lipid) as for example demonstrated by ORO staining. Lipid droplets in cells not exposed to myelin likely derive from lipid in the culture medium and/or apoptotic other macrophages in the culture. Primary macrophages may be used but also myeloid or monocyte-like cells or cell lines such as U937 (human): ATCC CRL-1593.2; THP-1 (human): ATCC TIB-202; RAW (mouse): ATCC TIB-71, or specific monocyte-like cells such as rodent, marmoset or human myeloid dendritic cells (mDC) or microglial cells can develop the foamy characteristics when fed lipid-rich fraction and are advantageously used in a method as provided herein.

We hypothesized that foamy macrophages in MS brain are anti-inflammatory M2-type macrophages as generated under laboratory conditions. We then hypothesized that foamy macrophages actively contribute to the resolution of brain inflammation. Our findings reveal an important and previously overlooked anti-inflammatory role for foamy macrophages in MS lesions.

Provided is the insight that multiple sclerosis (MS) lesion activity concurs with the extent of inflammation, demyelination and axonal suffering, in short, with the balance between local pro- and anti-inflammatory activities. Pro-inflammatory myeloid cells contribute to lesion development, but the self-limiting nature of lesions now is explained as earlier unidentified anti-inflammatory mechanisms. We show herein that lipid ingestion, and in particular myelin ingestion by myeloid cells induces a foamy appearance and confers anti-inflammatory function. We show that myelin-containing foam cells in MS lesions consistently express a series of anti-inflammatory molecules while mainly lacking pro-inflammatory cytokines. Unique location-dependent cytokine and membrane receptor expression profiles allow for functional specialization allowing for differential responses to micro-environmental cues. The invention therewith provides a novel, and advantageously an essentially human in vitro model of MS using foamy macrophages wherein it functionally is confirmed that in human macrophages myelin ingestion induces an anti-inflammatory program, to which program the effects of test compounds can be evaluated.

Also provided is novel insights into the mechanisms of lesion control and opens new roads to therapeutic intervention at the exact site where it most counts in MS, the recurrent inflammatory lesion in the brain.

### FURTHER EXAMPLES

The peptides LQGV (SEQ ID NO:1), MTR, MTRV (SEQ ID NO:20), AQGV (SEQ ID NO:2), LAGV (SEQ ID NO:10), AQG, LQG, VLPALPQ (SEQ ID NO:13), LAG, and VLPALP (SEQ ID NO:4) as mentioned herein were prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the alpha-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H₂O/triisopropylsilane (TIS) 95:2.5:2.5. After 30 minutes, TIS was added until decolorization. The solution was evaporated in vacuo and the peptide precipitated with diethyl ether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/minute; absorbance was detected from 190-370 nm. There were different gradient systems used. For example, for peptides LQG and LQGV: ten minutes 100% A followed by linear gradient 0-10% B in 50 minutes. For example for peptides VLPALP (SEQ ID NO:4) and VLPALPQ: five minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

### Abbreviations Used

LPS, lipopolysaccharide; IL, Interleukin; PGE prostaglandin E; EAE, experimental autoimmune encephalomyelitis; Th, T helper; ATCC American Type Culture Collection; IL-1ra, receptor antagonist; HLA human leukocyte antigen; TGF transforming growth factor; ELISA enzyme-linked immuno sorbent assay; COX cyclooxygenase; TNF, tumor necrosis factor; IFN interferon; MS Multiple sclerosis; CNS Central nervous system; NAWM Normal appearing white matter; MOG Myelin oligodendrocyte glycoprotein; ORO Oil red O;

TABLE I
Markers and antibodies.

| Molecule/marker | Function |
|---|---|
| IL-1ra | Anti-inflammatory, Endogenous IL-1 antagonist |
| IL-4 | Anti-inflammatory |
| PGES | Anti-inflammatory |
| TGF-beta | Anti-inflammatory |
| CCL18 | expressed by T/B cells, DC, macrophages, |
| | chemotactic to naïve T cells and iDC |
| HLA class II | Antigen presentation to CD4+ T cells |
| CD163 | Scavenger receptor for haptoglobin-hemoglobin |
| | complexes, anti-inflammatory actions |
| Mannose receptor | Lectin, recognition of micro-organisms |
| CD11b | Forms complement receptor 3 with CD18 |
| IL-1beta | Pro-inflammatory cytokine |
| TNF-alpha | Pro-inflammatory cytokine |
| IL-6 | Pro- and anti-inflammatory actions |
| IL-12 p40/p70 | Pro-inflammatory cytokine |
| MOG | Myelin oligodendrocyte glycoprotein |
| MAP-2 | Neuronal protein |

The invention is further explained with the aid of the following illustrative examples.

### Example 1

### Myelin-Laden Macrophages are Anti-Inflammatory Consistent with Foam Cells in Multiple Sclerosis Material and Methods

### Immunohistochemical Analysis of Postmortem MS Brain Tissue

Human autopsy brain tissue from 5 MS patients was provided by the Netherlands Brain Bank in Amsterdam. Immunohistochemistry was performed on frozen sections of MS brain tissue to detect expression of (anti-)inflammatory markers and CNS antigens (Table 1) as described previously (Hoefakker et al., 1995). In brief, 6 μm frozen sections were cut and thawed on to glass slides. Slides were kept overnight at room temperature in humidified atmosphere. After air-drying, slides were fixed in acetone containing 0.02% (v/v) H₂O₂. Slides were then air-dried for ten minutes, washed with PBS and incubated with optimally diluted primary antibody overnight at 4° C. in humidified atmosphere. Incubations with secondary rabbit anti-mouse-Ig-biotin (Dako) and tertiary horseradish peroxidase (HRP)-labeled avidin-biotin-complex (ABC/HRP: Dako) were performed for one hour at RT. HRP activity was revealed by incubation for ten minutes at RT with 3-amino-9-ethyl-carbazole (AEC: Sigma), leading to a bright red precipitate. After washing, sections were counterstained with hematoxylin, and embedded with glycerol-gelatin. Omission of primary antibody acted as control staining. Myelin degradation products were detected with oil-red O (ORO), which stains neutral lipids, as previously described (Chayen and Bitensky, 1991). The used antibodies were the anti-inflammatory markers IL-1ra (Biosource), IL-4 (U-Cytech), PGES (Cayman), TGF-beta (Santa Cruz), and CCL18 (R&D); for antigen recognition and presentation HLA class II (Dako), CD163, mannose receptor, CD11b (BD biosciences); as pro-inflammatory markers IL-1beta (gift from Dr. Boraschi), TNF-alpha (U-Cytech), IL-6 (Genzyme), IL-12p40/p70 (Pharmingen); for CNS proteins MOG, MAP-2 (Pierce).

### In Vitro Model for Myelin-Driven Foam Cell Formation

Myelin was isolated as described previously (Norton and Poduslo, 1973). In short, white matter derived from post-mortem brain tissue was homogenized in 0.32 M sucrose and subsequently layered on 0.85 M sucrose. After centrifugation at 75,000 g myelin was collected from the interface, washed in water and suspended in water for osmotic shock. Using this method, the purified myelin was shown to be free of any recognizable fragments of other subcellular elements. Previous studies have shown that purified myelin structurally resembled the whole multilamellar myelin structure surrounding as seen in tissue sections using electron microscopy (Autilio et al., 1964).

Peripheral blood mononuclear cells were isolated from heparinized blood from healthy donors using a Ficoll density gradient. Subsequently, monocytes were purified using Percoll density gradient resulting in >80% monocytes. Monocytes were cultured in suspension at a concentration of 1×10⁶ cells/ml in TEFLON® flasks (Nalgene) in RPMI with 5% human AB serum. After five to seven days monocyte-derived macrophages were recovered from the Teflon flasks and seeded in tissue culture plates. After 24 hours, non-adherent cells were removed and remaining cells were >95% macrophages as determined by macrophage-specific esterase staining. Foamy macrophages were generated in vitro by incubating macrophages with myelin for 24 hours to seven days (referred to as one day and seven day-old foamy macrophages). In most experiments 50 microg/ml myelin was used. Control macrophages were obtained from the same donor, and not fed with myelin.

### ELISA

To determine cytokine production in culture supernatants of foamy macrophages commercial capture ELISA was performed. TNF-alpha, IL-10 and IL-12p40 were measured in the collected culture supernatants. ELISA was performed according to the manufacturers' guidelines (Biosource). Briefly, polystyrene microtiter wells (Immuno Maxisorp) were coated overnight at 4° C. with monoclonal anti-cytokine capture antibodies. Wells were blocked for two hours at RT with PBS/0.5% BSA, followed by washing (0.9% NaCl/0.1% Tween20). Freshly thawed supernatants of the cell cultures and recombinant human cytokine-standards were incubated in duplicates for two hours at RT in the presence of a biotinylated second anti-cytokine detection antibody. After washing, wells were incubated with HRP-labeled poly-streptavidin (CLB) for 30 minutes at RT. HRP revelation was performed with 3,3′,5,5′-tetramethylbenzidine (TMB) peroxidase (KPL). Color development was stopped by adding equal volume of 1M H₂SO₄. Optical density was measured at 450 nm.

CCL18 levels were measured by sandwich ELISA assay using a commercially available CytoSet (Biosource), consisting of a capture-antibody, a biotinylated detection-antibody, recombinant CCL18 standard and streptavidin-HRP conjugate. Assay conditions were exactly as described by the manufacturer.

### Real-Time Quantitative PCR

To quantify mRNA expression by foamy macrophages total RNA was extracted from cell cultures using the GenElute Mammalian Total RNA kit (Sigma). RNA samples were treated with DNAse I (Invitrogen) to remove any contaminating DNA. Using 1 microg of the total RNA as template, copy DNA (cDNA) was prepared using the AMV Reverse Transcription System (Promega). To determine target gene mRNA expression, real-time quantitative reverse-transcription-PCR was performed using TaqMan technology (PE-Applied Biosystems) as described previously (van der Fits et al., 2003). Target gene expression levels were corrected for GAPDH mRNA levels. Sequences of the PCR primers (PE Biosystems), and fluorogenic probes (Eurogentec) are: forward primer 5′CCTTCCTCCTGTGCCTGATG (SEQ ID NO: ______), reverse primer 5′ACAATCTCATTTGAATCAGGAA (SEQ ID NO: ______), probe 5′TGCCCGACTCCCTTGGGTGTCA (SEQ ID NO: ______) for COX-2; forward primer 5′ACGGCGCTGTCATCGATT (SEQ ID NO: ______), reverse primer 5′GGCATTCTTCACCTGCTCCA (SEQ ID NO: ______), probe 5′CTTCCCTGTGAAAACAAGAGCAAGGCC (SEQ ID NO: ______) for IL-10; forward primer 5′GCCCAGGCAGTCAGATCATC (SEQ ID NO: ______), reverse primer 5′-GGGTTTGCTACAACATGGGCT (SEQ ID NO: ______), probe 5′CTCGAACCCCGAGTGACAAGCCTG (SEQ ID NO: ______) for TNF-α; forward primer 5′CACCGGAACGACATGGAGA (SEQ ID NO: ______), reverse primer 5′TCCAGGCGACAAAAGGGTTA (SEQ ID NO: ______), probe 5′TGGGCTTCGTCTACTCCTTTCTGGGTC (SEQ ID NO: ______) for PGES; forward primer 5′GCCTGGCCTCCAGAAAGACC (SEQ ID NO: ______), reverse primer 5′ACCTGGTACATCTTCAAGTCTTCATAAAT (SEQ ID NO: ______), probe 5′CTTTTATGATGGCCCTGTGCCTTAGT (SEQ ID NO: ______) for IL-12p35; forward primer 5′GCCAGGAGTTGTGAGTTTCCA (SEQ ID NO: ______), reverse primer 5′-TGCAAGGCCCTTCATGATG (SEQ ID NO: ______), probe 5′TCTGACCACTTCTCTGCCTGCCCA (SEQ ID NO: ______) for CCL18. forward primer 5′-GTTCCCCATATCCAGTGTGG (SEQ ID NO: ______), reverse primer 5′-TCCTTTGCAAGCAGAACTGA (SEQ ID NO: ______), probe TGGCTGTG (Roche) for IL-23p19.

### Statistical Analysis

Statistical analysis was performed using the non-parametric Mann-Whitney analysis. P values <0.05 were considered significant.

Multiple sclerosis (MS) is a chronic inflammatory autoimmune disease of the central nervous system (CNS) and is characterized by the presence of demyelinated areas throughout the CNS (Sospedra and Martin, 2005). Various mechanisms leading to demyelination and axonal suffering have been implicated and the production of toxic inflammatory mediators by infiltrating and resident CNS macrophages is believed to play a pivotal role (Becher et al., 2000; Cannella and Raine, 2004; Lassmann, 2004; Matute and Perez-Cerda, 2005; Raine, 1994; Sospedra and Martin, 2005; Wingerchuk et al., 2001).

Different subsets of myeloid cells have distinct roles in the development of experimental autoimmune encephalomyelitis (EAE), an animal model for MS. These distinct and specialized roles of myeloid cells depend on their origin and, importantly, their location (Greter et al., 2005; Heppner et al., 2005; McMahon et al., 2005; Platten and Steinman, 2005). As such, perivascular cells appear to be optimally positioned for the modulation of infiltrating T cell activity whereas parenchymal myeloid cells may have a more prominent role in mechanisms involved in myelin breakdown and axonal suffering (Platten and Steinman, 2005).

The plasticity and functional polarization of macrophages have received renewed attention in light of novel key properties of different forms of macrophages. Two extremes of a continuum have been identified for macrophages, being M1, or classically activated macrophages, and M2, or alternatively activated macrophages (Gordon, 2003; Mantovani et al., 2004; Mantovani et al., 2002; Mosser, 2003). The M1 phenotype is typically induced in vitro by IFN-gamma, TNF-alpha or LPS, whereas the M2 phenotype can be induced by IL-10, IL-4 or by the lipid mediator PGE₂, which is a strong inhibitor of pro-inflammatory immune responses (Gratchev et al., 2001; Harris et al., 2002; Hinz et al., 2000; Ikegami et al., 2001; Kalinski et al., 1997). M1 macrophages are characterized by a high production of pro-inflammatory mediators and are involved in Th1 cell responses and killing of micro-organisms and tumor cells.

In contrast, M2 macrophages are associated with Th2 responses, scavenging of debris, promotion of tissue remodeling and repair and expression of anti-inflammatory molecules, including IL-1ra (IL-1 receptor antagonist) and CCL18 (Gordon, 2003; Mantovani et al., 2004). CCL18 in particular is a specific marker for human alternatively activated macrophages (Goerdt et al., 1999; Gordon, 2003; Kodelja et al., 1998; Mantovani et al., 2002) and is likely involved in immune suppression. Demyelinating MS lesions are characterized by the presence of foamy macrophages, a characteristic subset of myeloid cells, which acquire their distinctive morphology by ingestion and accumulation of vast amounts of myelin-derived lipids. Foamy macrophages originate from both resident microglia and infiltrating monocytes. 30-80% of foamy macrophages in demyelinating lesions are estimated to be blood-derived (Li et al., 1996). Besides their apparent role in scavenging myelin, it is still poorly understood if and how foamy macrophages may affect the local inflammatory process. Since MS lesions are self-limiting and do not expand indefinitely it is likely that local mechanisms restrict CNS inflammation and may also promote tissue repair. We hypothesized that foamy macrophages are anti-inflammatory M2-type macrophages and actively contribute to the resolution of brain inflammation and hence to tissue integrity and function. Our findings reveal an important and previously overlooked anti-inflammatory and modulatory role for foamy macrophages in MS lesions.

### Results of Example 1

### Foamy Macrophages Express Anti-Inflammatory Markers and Demonstrate a Unique Location-Dependent Phenotype

To determine the immune phenotype of lipid-laden foamy macrophages in MS lesions, we used antibodies against CNS proteins, various surface markers involved in antigen recognition and presentation, and pro- and anti-inflammatory markers characteristic for M1 and M2 macrophages (Goerdt et al., 1999; Gordon, 2003; Kodelja et al., 1998; Mantovani et al., 2002). Foamy macrophages were defined by their characteristic morphology, strong HLA-DR expression and presence of neutral lipids, which are detected by oil red O histochemistry (ORO). To determine whether foamy macrophages display phenotypic and functional specialization dependent on micro-location, we analyzed the phenotype of these cells in different micro-locations. We distinguished between foamy macrophages within the lesion, in perivascular spaces within the lesion and in the outer or inner rim. The distinction between the outer and inner rim was based on the presence of neutral lipids, MOG and on the size of the foamy macrophages. Outer rim foamy macrophages were smaller in size and contained more MOG, but less neutral lipids than inner rim foamy macrophages.

IL-6, a cytokine with pro-as well as anti-inflammatory properties as well as the anti-inflammatory M2 marker IL-1ra and prostaglandin E₂ synthase (PGES) were differentially expressed in the distinct areas of an MS-lesion. Whereas IL-6 and IL-1ra were detected mostly in perivascular and lesional foamy macrophages, PGES was mostly expressed in the outer, and to a lesser extent in the inner rim. Importantly, expression patterns between cells varied even when cells were in close proximity. Mannose receptor, which is characteristic for M2 macrophages (Gordon, 2003; Mantovani et al., 2004; Mantovani et al., 2002; Mosser, 2003), was highly expressed on foamy macrophages in perivascular spaces but was mostly absent on parenchymal foamy macrophages. Occasionally, a weakly positive cell was observed which was always in the vicinity of a blood vessel. TGF-beta expression showed the reverse expression pattern with more pronounced expression by parenchymal foamy macrophages compared to perivascular foamy macrophages.

As hypothesized, the relative levels of expression were related to specific micro-locations within the lesion. Foamy macrophages in the lesion rim contained MOG, and immunoreactivity showed a decreasing trend towards the center of the lesion, possibly reflecting time-dependent myelin degradation. In contrast, intracellular neuronal antigen MAP-2 immunoreactivity increased towards the center of the lesion, implicating that neuronal damage occurs mostly in the lesion center. Only foamy macrophages within perivascular spaces expressed the surface markers CD11b, CD163 and mannose receptor. The anti-inflammatory molecules IL-1ra, CCL18, IL-10, TGF-beta and IL-4 were all strongly expressed by foamy macrophages, and expression was highest in the center of the lesion. Interestingly, IL-10 expression was absent on foamy macrophages in perivascular spaces. The pro-inflammatory cytokines TNF-alpha, IL-1beta, IL-12p40/70 were not expressed by foamy macrophages in any of the micro-locations, whereas cells associated with vessels in normal appearing white matter (NAWM) did express these pro-inflammatory cytokines. Phenotypic heterogeneity was not observed among non-foamy macrophages that were present in low numbers in perivascular spaces in NAWM.

Thus, we demonstrate that foamy macrophages in the brain have clear anti-inflammatory characteristics, resemble M2 macrophages, and have a unique phenotype depending on the micro-location.

Myelin Induces a Foamy Morphology in Macrophages Resembling that of Foamy Macrophages in Situ

Next, we set out to determine whether ingestion of myelin in vitro results in an anti-inflammatory function of foamy macrophages as observed in situ. Therefore, we first developed a fully human in vitro model of foamy macrophages. In short, human monocyte-derived macrophages are cultured in the absence or presence of human brain-derived myelin for 24 hours. Whereas cells cultured in the absence of myelin did not appear foamy (at magnification 32×), those cultured with myelin acquire a characteristic foamy morphology as observed by light microscopy. Human primary macrophages obtained from healthy donors were fed with 50 microg/ml human myelin and changes in the morphology were monitored by light microscopy and by ORO staining to detect intracellular neutral lipids. Although small individual changes in kinetics between individual donors were observed, macrophages acquired a foamy morphology between 24 and 48 hours and contained a markedly increased number and size of lipid droplets in comparison to control macrophages (i.e., not fed with myelin) as demonstrated by ORO staining. The typical foamy morphology of macrophages could still be observed one week upon the initial addition of myelin. Macrophage viability was not affected by myelin ingestion when a dose range of 1-100 microg/ml as was used, as was demonstrated by trypan blue staining.

### Foamy Macrophages do not Mount Pro-Inflammatory Responses to Prototypical Inflammatory Stimuli and Produce Anti-Inflammatory Mediators

To assess the effect of myelin ingestion on macrophage function, cytokine levels were determined in supernatants of myelin-laden macrophages before and after LPS stimulation. Since variation in myelin lipid composition between MS and normal brain has been reported (Woelk and Borri, 1973), myelin was isolated from white matter of three control brains and three MS brains to investigate possible functional differences. Macrophages were incubated with the distinct myelin preparations for 24 hours and IL-10 and IL-12p40 levels were determined in the supernatants by ELISA. None of the myelin preparations induced IL-12p40 and only the highest dose of one MS brain-derived myelin was associated with a transient IL-10 induction. All myelin preparations inhibited LPS-induced IL-12p40 and IL-10 induction in a dose-dependent fashion. No significant differences were observed in cytokine production between foamy macrophages generated using the different myelin preparations. For subsequent experiments 50 microg/ml myelin was used.

Next, the effect of myelin ingestion on LPS-induced mRNA levels of different pro- and anti-inflammatory mediators was determined. Macrophages were incubated with myelin for 24 hours and subsequently stimulated with LPS for an additional two hours, after which RNA was isolated and real time RT-PCR was performed for IL-12p35, TNF-alpha, IL-10, COX-2, PGES and CCL18. LPS-induced IL-12p35 and TNF-alpha expression by foamy macrophages was completely inhibited. IL-10 was slightly but not significantly induced by LPS in control macrophages as well as foamy macrophages. COX-2 was increased after LPS stimulation in control macrophages but this induction was not significantly inhibited in foamy macrophages. Foamy macrophages showed between 15-50 and 8-12-fold induction of CCL18 and PGES compared to control macrophages. Thus, myelin ingestion resulted in a differential modulation of LPS responses. LPS-induced IL-12p40 and TNF-alpha expression was strongly and significantly inhibited, IL-10 and COX-2 expression remained unaffected and the expression of anti-inflammatory CCL18 and PGES significantly increased.

To determine whether myelin ingestion results in long-term modulation of macrophage function, macrophages were incubated with myelin for the indicated time periods and real time RT-PCR was performed for IL-12p35, IL-10, PGES and CCL18. IL-10 mRNA was not detectable at any time point. After myelin uptake IL-12p35 expression was decreased, albeit not significantly, over time in comparison to control macrophages. In contrast to IL-12p35 both PGES and CCL18 were induced by myelin. Seven day-old foamy macrophages expressed ten- and 90-fold more PGES and CCL18 than control macrophages. IL-12p40, IL-10, and CCL18 levels were subsequently determined in supernatants of these foamy macrophages. CCL18 is constitutively produced by macrophages and production by foamy macrophages is increased at day 7 after myelin ingestion, paralleling the increased CCL18 mRNA expression by foamy macrophages. IL-12p40 and IL-10 were not detectable.

Subsequently we determined whether the aberrant LPS response persisted over time. Seven days after initial myelin ingestion foamy macrophages were stimulated with 1 ng/ml LPS for 24 hours and cytokine levels in the supernatant were determined by ELISA. LPS-induced IL-12p40 and IL-10 production by these foamy macrophages was abolished completely whereas CCL18 was significantly increased. In addition, responses to other prototypical pro-inflammatory stimuli such as peptidoglycan and zymosan were also completely abolished.

The relapsing-remitting nature of MS strongly suggests the presence of potent counter-regulatory mechanisms that keep the disease in check. One such mechanism may be the active control of inflammation in the CNS itself thus preventing infinite expansion of the demyelinating lesion. Inflammation and demyelination are responsible for at least short-term neurological symptoms. Inflammation probably contributes to axonal loss as neurons are more vulnerable to environmental insults when the protective myelin sheaths are destroyed and the axons exposed (Grigoriadis et al., 2004; Kuhlmann et al., 2002). It is therefore imperative that in the developing lesions the production of toxic molecules is halted and that inflammation is limited allowing for tissue repair (Sospedra and Martin, 2005).

Myelin-laden foamy macrophages are abundantly present in demyelinating lesions and although it is generally assumed that these cells contribute to inflammation, evidence for this is scarce (van der Laan et al., 1996). This lack of data on foamy macrophage function in MS is in sharp contrast with the increasing attention for foam cells in atherosclerosis (Greaves and Gordon, 2005) reporting potent immune-regulatory functions by lipids and lipid-induced molecules (Harris et al., 2002; Joseph et al., 2004; Joseph et al., 2003; Lawrence et al., 2002; Pettus et al., 2002). Lipid-laden cells are anti-inflammatory (Lawrence et al., 2002) and it was shown that low-density lipoprotein (LDL) uptake by macrophages inhibits TNF-induced TNF expression and induces IL-10 (Ares et al., 2002; Lo et al., 1999; Varadhachary et al., 2001). Foamy macrophages in the rim of active demyelinating lesions have been shown to contain plasma LDL (Newcombe et al., 1994).

Here, we establish that foamy macrophages in active MS lesions have consistent immunosuppressive function, while displaying a unique surface phenotype dependent on the micro-location. In addition, we demonstrate that ingestion of human myelin alters human macrophage function in vitro by inducing anti-inflammatory molecules and by inhibiting responses to pro-inflammatory stimuli. The results presented here reveal a new regulatory pathway in MS.

We show here that the observed functional phenotype of foamy macrophages in MS lesions results from the accumulation of lipids derived from myelin and phagocytosed apoptotic cell membranes, in concert with local microenvironmental cues, such as differences in extracellular matrix content in the perivascular infiltrate versus the lesion in the brain parenchyma. Foamy macrophages demonstrate a phenotype resembling that of anti-inflammatory M2 macrophages, are likely to contribute to resolution of inflammation, and may therefore be responsible for inhibiting further lesion development and promoting lesion repair. In addition, they may also function as a first line of defense against infiltrating inflammatory myeloid cells. Future studies are required to elucidate which lipid components are able to regulate macrophage function and which mechanisms are involved. Understanding the mechanisms behind naturally occurring counter-regulatory processes allows for definition of new cellular targets for therapeutic drug design for the treatment of MS and even has broader applications for other foam cell-associated diseases including atherosclerosis and lung-conditions.

### Example 2

### Determining Whether Compounds Modulate Responses by Macrophages and Foam Cells Experimental Design

Human monocyte-derived macrophages were cultured in medium (=macrophages) or in the presence of human brain-derived myelin for 48 hours (=foam cells).

Macrophages and foam cells were cultured in the presence of 10 microg/ml compounds LAGV (SEQ ID NO:10), AQGV (SEQ ID NO:2), LAG, AQG, MTR, MTRV (SEQ ID NO:20), VLPALPQ (SEQ ID NO:13), VLPALP (SEQ ID NO:4), LQGV (SEQ ID NO:1), LQG (see for example PCT International Publication No. WO 03/029292 A2 (published Apr. 10, 2003), PCT International Publication No. WO 01/72831 A2 (published Oct. 4, 2001), PCT/EP2004/003747, and U.S. Patent Application Publications 20020064501 A1 (published May 30, 2002), 20030119720 A1 (published Jun. 26, 2003), 20030113733 A1 (published Jun. 19, 2003), US 2003/0220259 A1 (published Nov. 27 h, 2003) and 20030166556 A1 (published Sep. 4, 2003), the contents of all of which are incorporated by this reference) for three hours.

10 ng/ml LPS was added to the cultures for an additional 16 hours.

Supernatants were collected and ELISA performed for TNF-alpha, IL-12p40, and IL-10.

### Results:

Protein levels are depicted in Table 2.

LPS induced TNF-alpha, IL-12p40 and IL-10 in macrophages as expected, confirming the experimental system performed as usual.

Foam cells demonstrated decreased LPS responses for IL-10 and IL-12p40 as expected. LPS-induced TNF-alpha production by foam cells was not affected as has been observed before.

Effects of compounds on LPS responses are shown in Table 1.

The compounds did not affect macrophage or foam cell morphology or viability as judged by microscopic examination.

### Example 3

### Determining Whether Compounds Affect Cytokine Production by Human Macrophages and Foam Cells

### Experimental Design:

Human monocyte-derived macrophages from a healthy blood bank donor were cultured in medium (=macrophages) or in the presence of human brain-derived myelin for 48 hours (=foam cells).

Macrophages and foam cells were cultured in duplicate in the presence of 10 microg/ml of compounds LAGV (SEQ ID NO:10), AQGV (SEQ ID NO:2), LAG, AQG, MTR, MTRV (SEQ ID NO:20), VLPALPQ (SEQ ID NO:13), VLPALP (SEQ ID NO:4), LQGV (SEQ ID NO:1), LQG for two or eight hours, or cultured in macrophage medium with vehicle.

Cells were lysed and real time RT-PCR (TaqMan technology) was performed on all samples for GAPDH (housekeeping gene), TNF-alpha (pro-inflammatory), IL-12p35 (pro-inflammatory), IL-10 (anti-inflammatory), CCL18 (chemokine), COX-2 (prostaglandin pathway).

### Results:

Effects of compounds on mRNA expression levels are depicted in Tables 2, 3 and 4.

The compounds did not affect macrophage or foam cell morphology or viability as judged by microscopic examination.

cDNA quality of two samples was not sufficient for reliable semi-quantification. Values of these samples (#6, peptides two hours on macrophages; # 10, peptides eight hours on foam cells) have been omitted.

### Example 4

### Determining Whether Foam Cells Differentially Express Chemokines Compared to Control Macrophages

### Experimental Design:

Human monocyte-derived macrophages from a healthy blood bank donor were cultured in medium (=macrophages) or in the presence of human brain-derived myelin for 48 hours (=foam cells)

0 microg/ml of the compounds LAGV (SEQ ID NO:10), AQGV (SEQ ID NO:2) or LQGV (SEQ ID NO:1) is added to macrophages or foam cells for six hours, or macrophages or foam cells are cultured in macrophage medium with vehicle.

Cells were lysed, RNA isolated and Affymetrix microarray (U133+2 chip with 53.675 transcripts) was used according to the manufacturers instructions to determine relative mRNA levels

### Results:

Effect of myelin ingestion and additional effect of the compounds LAGV (SEQ ID NO:10), AQGV (SEQ ID NO:2) or LQGV (SEQ ID NO:1) on eight different selected chemokines is depicted in Table 6.

Compounds did not affect the chemokine expression by control macrophages.

### Example 5

The efficiency of ten different peptides was testes in four assays (as described in Examples 1 and 2). Peptides were ranked in order of efficiency, with one being the effect considered to be most beneficial for MS patients (i.e., low TNF, high IL-10, high CCL18) and ten being the most detrimental (i.e., high TNF, low IL-10, low CCL18). For each peptide, the mean ranking was calculated. The peptide with the highest overall ranking (i.e., the lowest number) is the most potent peptide with regards to the induction of effects which can be considered beneficial for MS patients. Results are shown in Table 7.

TABLE 2

LPS-induced cytokine responses by compounds
tested in human macrophages and in foam cells

| TNF-alpha | mean | Mean | sd | sd |
|---|---|---|---|---|
| (pg/ml) | macro- | foam | macro- | foam |
| compound | phages | cells | phages | cells |
| None | 21664 | 29366 | 2532 | 2733 |
| LAGV (SEQ | 8336 | 16464 | 322 | 2331 |
| ID NO:10) | | | | |
| AQGV (SEQ | 9075 | 15895 | 1688 | 723 |
| ID NO:2) | | | | |
| LAG | 13281 | 15895 | 2009 | 5225 |
| AQGV (SEQ | 14475 | 14531 | 482 | 563 |
| ID NO:2) | | | | |
| MTR | 13054 | 15839 | 2170 | 5626 |
| MTRV (SEQ | 14816 | 19249 | 3697 | 804 |
| ID NO:20) | | | | |
| VLPALPQ | 13167 | 20101 | 402 | 563 |
| (SEQ ID | | | | |
| NO:13) | | | | |
| VLPALP | 10723 | 20670 | 4823 | 6189 |
| (SEQ ID | | | | |
| NO:4) | | | | |
| LQGV (SEQ | 5381 | 13565 | 1125 | 482 |
| ID NO:1) | | | | |
| LQG | 4812 | 16691 | 643 | 884 |
| IL-b | mean | Mean | sd | sd |
| (pg/ml) | macro- | foam | macro- | foam |
| compound | phages | cells | phages | cells |
| None | 4140 | 485 | 118 | 29 |
| LAGV (SEQ | 2203 | 222 | 358 | 29 |
| ID NO:10) | | | | |
| AQGV (SEQ | 2977 | 186 | 105 | 0 |
| ID NO:2) | | | | |
| LAG | 2793 | 191 | 65 | 2 |
| AQGV (SEQ | 2078 | 179 | 110 | 0 |
| ID NO:2) | | | | |
| MTR | 2399 | 206 | 18 | 2 |
| MTRV (SEQ | 3105 | 235 | 251 | 11 |
| ID NO:20) | | | | |
| VLPALPQ | 3004 | 229 | 107 | 16 |
| (SEQ ID | | | | |
| NO:13) | | | | |
| VLPALP | 2654 | 153 | 11 | 20 |
| (SEQ ID | | | | |
| NO:4) | | | | |
| LQGV (SEQ | 3654 | 572 | 96 | 13 |
| ID NO:1) | | | | |
| LQG | 4209 | 601 | 172 | 22 |

| IL-12p40 | mean | Mean |
|---|---|---|
| (pg/ml) | macro- | foam |
| compound | phages | cells |
| None | 2258 | 1320 |
| LAGV (SEQ | 2038 | 1100 |
| ID NO:10) | | |
| AQGV (SEQ | 1527 | 1093 |
| ID NO:2) | | |
| LAG | 1799 | 899 |
| AQGV (SEQ | 1942 | 997 |
| ID NO:2) | | |
| MTR | 1910 | 912 |
| MTRV (SEQ | 2325 | 804 |
| ID NO:20) | | |
| VLPALPQ | 1901 | 1218 |
| (SEQ ID | | |
| NO:13) | | |
| VLPALP | 2426 | 1284 |
| (SEQ ID | | |
| NO:4) | | |
| LQGV (SEQ | 3364 | 1274 |
| ID NO:1) | | |
| LQG | 1859 | 1742 |

TABLE 3
Taqman results

| | |
|---|---|
| CCL18 | CCL18 |
| peptide treatment 2 hrs | peptide treatment 8 hrs |

| | | | |
|---|---|---|---|
| mean | s.d. | mean | s.d. |

| | | | | | | |
|---|---|---|---|---|---|---|
| macrophages | None | 0.99 | 0.45 | None | 1.14 | 0.29 |
| | LAGV | 1.00 | 0.13 | LAGV | 1.28 | 0.50 |
| | (SEQ ID | | | (SEQ ID | | |
| | NO:10) | | | NO:10) | | |
| | AQGV | 1.11 | 0.39 | AQGV | 2.42 | 0.16 |
| | (SEQ ID | | | (SEQ ID | | |
| | NO:2) | | | NO:2) | | |
| | LAG | 1.70 | 0.52 | LAG | 1.29 | 0.14 |
| | AQG | 1.00 | 0.43 | AQG | 1.19 | 0.25 |
| | MTR | ND | | MTR | 1.46 | 0.20 |
| | MTRV | 1.74 | 0.14 | MTRV | 1.13 | 0.39 |
| | (SEQ ID | | | (SEQ ID | | |
| | NO:20) | | | NO:20) | | |
| | VLPALPQ | 2.50 | 0.32 | VLPALPQ | 0.98 | 0.48 |
| | (SEQ ID | | | (SEQ ID | | |
| | NO:13) | | | NO:13) | | |
| | VLPALP | 1.31 | 0.20 | VLPALP | 2.20 | 0.48 |
| | (SEQ ID | | | (SEQ ID | | |
| | NO:4) | | | NO:4) | | |
| | LQGV | 2.41 | 1.04 | LQGV | 2.29 | 0.53 |
| | (SEQ ID | | | (SEQ ID | | |
| | NO:1) | | | NO:1) | | |
| | LQG | 1.33 | 0.31 | LQG | 1.91 | 0.10 |
| foam cells | None | 278.87 | 99.48 | None | 21.07 | 3.08 |
| | LAGV | 886.13 | 353.99 | LAGV | 81.82 | 9.06 |
| | (SEQ ID | | | (SEQ ID | | |
| | NO:10) | | | NO:10) | | |
| | AQGV | 600.00 | 211.58 | AQGV | 52.94 | 11.11 |
| | (SEQ ID | | | (SEQ ID | | |
| | NO:2) | | | NO:2) | | |
| | LAG | 219.51 | 33.08 | LAG | 92.32 | 31.57 |
| | AQG | 355.94 | 81.34 | AQG | 118.82 | 29.83 |
| | MTR | 262.81 | 65.57 | MTR | 124.05 | 24.11 |
| | MTRV | 277.78 | 94.19 | MTRV | 42.65 | 0.00 |
| | (SEQ ID | | | (SEQ ID | | |
| | NO:20) | | | NO:20) | | |
| | VLPALPQ | 205.43 | 46.94 | VLPALPQ | 49.25 | 3.90 |
| | (SEQ ID | | | (SEQ ID | | |
| | NO:13) | | | NO:13) | | |
| | VLPALP | 278.99 | 2.01 | VLPALP | 55.00 | 16.88 |
| | (SEQ ID | | | (SEQ ID | | |
| | NO:4) | | | NO:4) | | |
| | LQGV | 488.87 | 38.70 | LQGV | ND | |
| | (SEQ ID | | | (SEQ ID | | |
| | NO:1) | | | NO:1) | | |
| | LQG | 153.76 | 8.86 | LQG | 32.86 | 8.42 |

TABLE 4
Taqman results

| | |
|---|---|
| COX-2 | COX-2 |
| peptide treatment 2 hrs | peptide treatment 8 hrs |

| | | | |
|---|---|---|---|
| mean | s.d. | mean | s.d. |

| | | | | | | |
|---|---|---|---|---|---|---|
| macrophages | None | 0.591211 | 0.226438 | None | 0.94 | 0.32 |
| | LAGV | 1.28552 | 0.590098 | LAGV | 1.47 | 1.33 |
| | (SEQ ID | | | (SEQ ID | | |
| | NO:10) | | | NO:10) | | |
| | AQGV | 1.009161 | 0.171062 | AQGV | 0.71 | 0.03 |
| | (SEQ ID | | | (SEQ ID | | |
| | NO:2) | | | NO:2) | | |
| | LAG | 1.047836 | 0.344225 | LAG | 1.42 | 0.10 |
| | AQG | 1.28199 | 0.434554 | AQG | 1.58 | |
| | MTR | ND | | MTR | 2.40 | 1.05 |
| | MTRV | 1.321293 | 0.966936 | MTRV | 0.71 | 0.39 |
| | (SEQ ID | | | (SEQ ID | | |
| | NO:20) | | | NO:20) | | |
| | VLPALPQ | 1.01643 | 0.301701 | VLPALPQ | 0.50 | 0.15 |
| | (SEQ ID | | | (SEQ ID | | |
| | NO:13) | | | NO:13) | | |
| | VLPALP | 1.03924 | 0.186638 | VLPALP | 0.65 | 0.10 |
| | (SEQ ID | | | (SEQ ID | | |
| | NO:4) | | | NO:4) | | |
| | LQGV | 0.577613 | 0.104948 | LQGV | 0.67 | 0.12 |
| | (SEQ ID | | | (SEQ ID | | |
| | NO:1) | | | NO:1) | | |
| | LQG | 0.673839 | 0.100383 | LQG | 2.27 | 2.00 |
| foam cells | None | 0.89199 | 0.159893 | None | 0.79 | 0.09 |
| | LAGV | 0.912541 | 0 | LAGV | 2.19 | 0.46 |
| | (SEQ ID | | | (SEQ ID | | |
| | NO:10) | | | NO:10) | | |
| | AQGV | 0.763449 | 0.203646 | AQGV | 2.31 | 0.28 |
| | (SEQ ID | | | (SEQ ID | | |
| | NO:2) | | | NO:2) | | |
| | LAG | 0.722072 | 0.2582 | LAG | 1.66 | 0.50 |
| | AQG | 1.081277 | 0.053871 | AQG | 1.77 | 0.30 |
| | MTR | 0.73216 | 0.14501 | MTR | 2.48 | 0.01 |
| | MTRV | 1.445971 | 0 | MTRV | 2.15 | 0.53 |
| | (SEQ ID | | | (SEQ ID | | |
| | NO:20) | | | NO:20) | | |
| | VLPALPQ | 0.690174 | 0 | VLPALPQ | 1.15 | 0.07 |
| | (SEQ ID | | | (SEQ ID | | |
| | NO:13) | | | NO:13) | | |
| | VLPALP | 1.013483 | 0.109235 | VLPALP | 1.44 | 1.46 |
| | (SEQ ID | | | (SEQ ID | | |
| | NO:4) | | | NO:4) | | |
| | LQGV | 0.805138 | 0.046792 | LQGV | ND | |
| | (SEQ ID | | | (SEQ ID | | |
| | NO:1) | | | NO:1) | | |
| | LQG | 0.556831 | 0.159653 | LQG | 0.88 | 0.63 |

TABLE 5
Taqman results

| | |
|---|---|
| IL-10 | IL-10 |
| peptide treatment 2 hrs | peptide treatment 8 hrs |

| | | | |
|---|---|---|---|
| mean | s.d. | mean | s.d. |

| | | | | | | |
|---|---|---|---|---|---|---|
| macrophages | None | 0.838719 | 0.084748 | None | 0.96 | 0.23 |
| | LAGV | 0.93201 | 0.073727 | LAGV | 1.25 | 0.31 |
| | (SEQ ID | | | (SEQ ID | | |
| | NO:10) | | | NO:10) | | |
| | AQGV | 1.078281 | 0.162801 | AQGV | 1.97 | 0.17 |
| | (SEQ ID | | | (SEQ ID | | |
| | NO:2) | | | NO:2) | | |
| | LAG | 0.949998 | 0.155105 | LAG | 1.14 | 0.10 |
| | AQG | 0.794645 | 0.120295 | AQG | 1.28 | 0.23 |
| | MTR | ND | | MTR | 1.56 | 0.77 |
| | MTRV | 0.799543 | 0.059873 | MTRV | 0.83 | 0.20 |
| | (SEQ ID | | | (SEQ ID | | |
| | NO:20) | | | NO:20) | | |
| | VLPALPQ | 0.95685 | 0.180639 | VLPALPQ | 1.29 | 0.46 |
| | (SEQ ID | | | (SEQ ID | | |
| | NO:13) | | | NO:13) | | |
| | VLPALP | 0.79994 | 0.060407 | VLPALP | 1.39 | 0.25 |
| | (SEQ ID | | | (SEQ ID | | |
| | NO:4) | | | NO:4) | | |
| | LQGV | 0.623505 | 0.04854 | LQGV | 0.81 | 0.20 |
| | (SEQ ID | | | (SEQ ID | | |
| | NO:1) | | | NO:1) | | |
| | LQG | 0.604754 | | LQG | 1.91 | 0.80 |
| foam cells | None | 0.680602 | 0.118096 | None | 0.471046 | 0.074507 |
| | LAGV | 0.628717 | 0.031388 | LAGV | 1.34 | 0.19 |
| | (SEQ ID | | | (SEQ ID | | |
| | NO:10) | | | NO:10) | | |
| | AQGV | 0.788278 | 0.120264 | AQGV | 1.34 | 0.42 |
| | (SEQ ID | | | (SEQ ID | | |
| | NO:2) | | | NO:2) | | |
| | LAG | 0.564875 | 0.022564 | LAG | 0.91 | 0.10 |
| | AQG | 0.724893 | 0.06027 | AQG | 1.16 | 0.16 |
| | MTR | 0.801344 | 0.047998 | MTR | 0.99 | 0.05 |
| | MTRV | 0.936911 | 0.309266 | MTRV | 1.07 | 0.12 |
| | (SEQ ID | | | (SEQ ID | | |
| | NO:20) | | | NO:20) | | |
| | VLPALPQ | 0.56192 | 0.028053 | VLPALPQ | 1.01 | 0.17 |
| | (SEQ ID | | | (SEQ ID | | |
| | NO:13) | | | NO:13) | | |
| | VLPALP | 0.745603 | 0.103929 | VLPALP | 0.83 | 0.05 |
| | (SEQ ID | | | (SEQ ID | | |
| | NO:4) | | | NO:4) | | |
| | LQGV | 0.682842 | 0.090676 | LQGV | ND | |
| | (SEQ ID | | | (SEQ ID | | |
| | NO:1) | | | NO:1) | | |
| | LQG | 0.518613 | 0.032787 | LQG | 1.01 | 0.04 |

TABLE 6
Fold differences of selected chemokines
as determined by Affymetrix microarray

| | | Additional | Additional | Additional |
|---|---|---|---|---|
| | Effect of | effect of | effect of | effect of |
| Chemo- | myelin | LAGV (SEQ | AQGV (SEQ | LQGV (SEQ |
| kine | ingestion | ID NO: 10) | ID NO: 2) | ID NO: 1) |
| CCL2 | −1.6 | 3.27 | 5.3 | 3.1 |
| CCL3 | 2.5 | 1.1 | 1.1 | 1.0 |
| CCL4 | 3.3 | 1.3 | 1.4 | 1.1 |
| CCL5 | 4.3 | 1.4 | 2.1 | 1.7 |
| CCL7 | 1.6 | 1.6 | 2.5 | 1.9 |
| CXCL3 | 1.0 | 1.8 | 2.3 | 1.9 |
| CXCL8 | 5.1 | 1.5 | 2.0 | 1.8 |
| CCL18 | 3 | 2.8 | 4.5 | 3.3 |

TABLE 7a

Ranking of peptides as to suitability for
treatment of MS

| | | | | |
|---|---|---|---|---|
| mean | | | | |
| ranking | ranking | ranking | ranking | ranking |
| | a | b | c | d |

| | | | | | |
|---|---|---|---|---|---|
| LQGV | 2.25 | 1 | 2 | 3 | 3 |
| (SEQ ID | | | | | |
| NO:1) | | | | | |
| MTR | 4.25 | 3 | 6 | 1 | 7 |
| MTRV | 5.25 | 8 | 3 | 4 | 6 |
| (SEQ ID | | | | | |
| NO:20) | | | | | |
| AQGV | 5.5 | 4 | 8 | 8 | 2 |
| (SEQ ID | | | | | |
| NO:2) | | | | | |
| LAGV | 5.5 | 6 | 5 | 10 | 1 |
| (SEQ ID | | | | | |
| NO: 10) | | | | | |
| AQG | 5.75 | 2 | 9 | 9 | 3 |
| LQG | 6 | 7 | 1 | 6 | 10 |
| VLPALPQ | 6 | 9 | 4 | 2 | 9 |
| (SEQ ID | | | | | |
| NO:13) | | | | | |
| LAG | 6.25 | 5 | 7 | 5 | 8 |
| VLPALP | 8 | 10 | 10 | 7 | 5 |
| (SEQ ID | | | | | |
| NO:4) | | | | | |

TABLE 7b
Ranking parameters

| | |
|---|---|
| A | lowest TNF foamy macrophages |
| B | highest IL-10 foamy macrophages |
| C | highest CCL18 macrophages |
| D | highest CCL18 foamy macrophages |

### REFERENCES

Ares M. P., M. Stollenwerk, A. Olsson, B. Kallin, S. Jovinge, and J. Nilsson. Decreased inducibility of TNF expression in lipid-loaded macrophages. BMC Immunol. 2002; 3:13.
Autilio L. A., W. T. Norton, and R. D. Terry. The Preparation and Some Properties of Purified Myelin from the Central Nervous System. J. Neurochem. 1964; 11:17-27.
Becher B., A. Prat, J. P. Antel. Brain-immune connection: immuno-regulatory properties of CNS-resident cells. Glia 2000; 29:293-304.
Cannella B. and C. S. Raine. Multiple sclerosis: Cytokine receptors on oligodendrocytes predict innate regulation. Ann. Neurol. 2004; 55:46-57.
Chayen J. and L. Bitensky. Analysis of chemical components of cells and tissues; reactions for lipids. Practical Histochemistry. West Sussex: Wiley, 1991; 45.
Goerdt S., O. Politz, K. Schledzewski, R. Birk, A. Gratchev, P. Guillot, et al. Alternative versus classical activation of macrophages. Pathobiology 1999; 67:222-6.
Gordon S. Alternative activation of macrophages. Nat. Rev. Immunol. 2003; 3:23-35.
Gratchev A., K. Schledzewski, P. Guillot, and S. Goerdt. Alternatively activated antigen-presenting cells: molecular repertoire, immune regulation, and healing. Skin Pharmacol. Appl. Skin Physiol. 2001; 14:272-9.
Greaves D. R. and S. Gordon. Thematic review series: The Immune System and Atherogenesis. Recent insights into the biology of macrophage scavenger receptors. J. Lipid Res. 2005; 46:11-20.
Greter M., F. L. Heppner, M. P. Lemos, B. M. Odermatt, N. Goebels, T. Laufer, et al. Dendritic cells permit immune invasion of the CNS in an animal model of multiple sclerosis. Nat. Med. 2005; 11:328-34.
Grigoriadis N., T. Ben-Hur, D. Karussis, and I. Milonas. Axonal damage in multiple sclerosis: a complex issue in a complex disease. Clin. Neurol. Neurosurg. 2004; 106:211-7.
Harris S. G., J. Padilla, L. Koumas, D. Ray, and R. P. Phipps. Prostaglandins as modulators of immunity. Trends Immunol. 2002; 23:144-50.
Heppner F. L., M. Greter, D. Marino, J. Falsig, G. Raivich, N. Hovelmeyer, et al. Experimental autoimmune encephalomyelitis repressed by microglial paralysis. Nat. Med. 2005; 11:146-52.
Hinz B., K. Brune, and A. Pahl. Prostaglandin E(2) up-regulates cyclooxygenase-2 expression in lipopolysaccharide-stimulated RAW 264.7 macrophages. Biochem. Biophys. Res. Commun. 2000; 272:744-8.
Hoefakker S., W. J. Boersma, and E. Claassen. Detection of human cytokines in situ using antibody and probe based methods. J. Immunol. Methods 1995; 185:149-75.
Ikegami R., Y. Sugimoto, E. Segi, M. Katsuyama, H. Karahashi, F. Amano, et al. The expression of prostaglandin E receptors EP2 and EP4 and their different regulation by lipopolysaccharide in C3H/HeN peritoneal macrophages. J. Immunol. 2001; 166:4689-96.
Joseph S. B., M. N. Bradley, A. Castrillo, K. W. Bruhn, P. A. Mak, L. Pei, et al. LXR-dependent gene expression is important for macrophage survival and the innate immune response. Cell 2004; 119:299-309.
Joseph S. B., A. Castrillo, B. A. Laffitte, D. J. Mangelsdorf, and P. Tontonoz. Reciprocal regulation of inflammation and lipid metabolism by liver X receptors. Nat. Med. 2003; 9:213-9.
Kalinski P., C. M. Hilkens, A. Snijders, F. G. Snijdewint, and M. L. Kapsenberg. Dendritic cells, obtained from peripheral blood precursors in the presence of PGE2, promote Th2 responses. Adv. Exp. Med. Biol. 1997; 417:363-7.
Kodelja V., C. Muller, O. Politz, N. Hakij, C. E. Orfanos, and S. Goerdt. Alternative macrophage activation-associated CC-chemokine-1, a novel structural homologue of macrophage inflammatory protein-1 alpha with a Th2-associated expression pattern. J. Immunol. 1998; 160:1411-8.
Kuhlmann T., G. Lingfeld, A. Bitsch, J. Schuchardt, and W. Bruck. Acute axonal damage in multiple sclerosis is most extensive in early disease stages and decreases over time. Brain 2002; 125:2202-12.
Lassmann H. Recent neuropathological findings in MS—implications for diagnosis and therapy. J. Neurol. 2004; 251 Suppl 4:IV2-5.
Lawrence T., D. A. Willoughby, and D. W. Gilroy. Anti-inflammatory lipid mediators and insights into the resolution of inflammation. Nat. Rev. Immunol. 2002; 2:787-95.
Li H., M. L. Cuzner, and J. Newcombe. Microglia-derived macrophages in early multiple sclerosis plaques. Neuropathol. Appl. Neurobiol. 1996; 22:207-15.
Lo C. J., M. Fu, F. R. Lo, and H. G. Cryer. Macrophage TNF mRNA expression induced by LPS is regulated by sphingomyelin metabolites. Shock 1999; 11:411-5.
Mantovani A., A. Sica, S. Sozzani, P. Allavena, A. Vecchi, and M. Locati. The chemokine system in diverse forms of macrophage activation and polarization. Trends Immunol. 2004; 25:677-86.
Mantovani A., S. Sozzani, M. Locati, P. Allavena, and A. Sica. Macrophage polarization: tumor-associated macrophages as a paradigm for polarized M2 mononuclear phagocytes. Trends Immunol. 2002; 23:549-55.
Matute C. and F. Perez-Cerda. Multiple sclerosis: novel perspectives on newly forming lesions. Trends Neurosci. 2005; 28:173-5.
McMahon E. J., S. L. Bailey, C. V. Castenada, H. Waldner, and S. D. Miller. Epitope spreading initiates in the CNS in two mouse models of multiple sclerosis. Nat. Med. 2005; 11:335-339.
Mosser D. M. The many faces of macrophage activation. J. Leukoc. Biol. 2003; 73:209-12.
Newcombe J., H. Li, and M. L. Cuzner. Low density lipoprotein uptake by macrophages in multiple sclerosis plaques: implications for pathogenesis. Neuropathol. Appl. Neurobiol. 1994; 20:152-62.
Norton W. T. and S. E. Poduslo. Myelination in rat brain: method of myelin isolation. J. Neurochem. 1973; 21:749-57.
Pettus B. J., C. E. Chalfant, and Y. A. Hannun. Ceramide in apoptosis: an overview and current perspectives. Biochim. Biophys. Acta. 2002; 1585:114-25.
Platten M. and L. Steinman. Multiple sclerosis: trapped in deadly glue. Nat. Med. 2005; 11:252-3.
Raine C. S. Multiple sclerosis: immune system molecule expression in the central nervous system. J. Neuropathol. Exp. Neurol. 1994; 53:328-37.
Sospedra M. and R. Martin. Immunology of multiple sclerosis *. Annu. Rev. Immunol. 2005; 23:683-747.
van der Fits L., L. I. van der Wel, J. D. Laman, E. P. Prens, and M. C. Verschuren. Psoriatic lesional skin exhibits an aberrant expression pattern of interferon regulatory factor-2 (IRF-2). J. Pathol. 2003; 199:107-14.
van der Laan L. J., S. R. Ruuls, K. S. Weber, I. J. Lodder, E. A. Dopp, and C. D. Dijkstra. Macrophage phagocytosis of myelin in vitro determined by flow cytometry: phagocytosis is mediated by CR3 and induces production of tumor necrosis factor-alpha and nitric oxide. J. Neuroimmunol. 1996; 70:145-52.
Varadhachary A. S., M. Monestier, and P. Salgame. Reciprocal induction of IL-10 and IL-12 from macrophages by low-density lipoprotein and its oxidized forms. Cell. Immunol. 2001; 213:45-51.
Vulcano M., S. Struyf, P. Scapini, M. Cassatella, S. Bemasconi, R. Bonecchi, et al. Unique regulation of CCL18 production by maturing dendritic cells. J. Immunol. 2003; 170:3843-9.
Wingerchuk D. M., C. F. Lucchinetti, and J. H. Noseworthy. Multiple sclerosis: current pathophysiological concepts. Lab. Invest. 2001; 81:263-81.
Woelk H. and P. Borri. Lipid and fatty acid composition of myelin purified from normal and MS brains. Eur. Neurol. 1973; 10:250-60.

### FURTHER REFERENCES

WO 99/59671,
WO 01/72831,
WO 97/49721,
WO 01/10907,
WO 01/11048.

The contents of the entirety of each reference identified herein is incorporated in its entirety by this reference.

What is claimed is:

1. A method for modulating an inflammatory neurological disorder in a subject, said method comprising:
providing the subject with a peptide selected from the group consisting of LQGV (SEQ ID NO:1), MTR, MTRV (SEQ ID NO:20), AQGV (SEQ ID NO:2), LAGV (SEQ ID NO:10), AQG, LQG, VLPALPQ (SEQ ID NO:13), LAG, and VLPALP (SEQ ID NO:4).

2. The method according to claim 1, wherein said peptide is selected from the group consisting of LQGV (SEQ ID NO:1), MTR, MTRV (SEQ ID NO:20), AQGV (SEQ ID NO:2), LAGV (SEQ ID NO:10), AQG, and LQG.

3. The method according to claim 2, wherein said peptide is selected from the group consisting of LQGV (SEQ ID NO:1), LAGV (SEQ ID NO:10), and AQGV (SEQ ID NO:2).

4. The method according to claim 2, wherein said peptide is selected from the group consisting of LQGV (SEQ ID NO:1), MTR, and MTRV (SEQ ID NO:20).
